SEQUENCE LISTING (26 sequences)

SEQ ID NO: 1 - DNA, 19 bp, Artificial Sequence
  PCR primer
cctccagcagatgcaagaa

SEQ ID NO: 2 - DNA, 20 bp, Artificial Sequence
  PCR primer
gcttgcacttcatcctttgg

SEQ ID NO: 3 - DNA, 20 bp, Artificial Sequence
  PCR primer
tctgtggtcaagtccgaggc

SEQ ID NO: 4 - DNA, 20 bp, Artificial Sequence
  PCR primer
ttctccagttcgcagtccag

SEQ ID NO: 5 - DNA, 24 bp, Artificial Sequence
  PCR primer
ggaagaaatgctgaaggtggagac

SEQ ID NO: 6 - DNA, 22 bp, Artificial Sequence
  PCR primer
agtccccatccccttcaatagc

SEQ ID NO: 7 - DNA, 21 bp, Artificial Sequence
  PCR primer
ccaacgagaagagtatgaggc

SEQ ID NO: 8 - DNA, 21 bp, Artificial Sequence
  PCR primer
gtgcttttaatccctcctcag

SEQ ID NO: 9 - DNA, 23 bp, Artificial Sequence
  PCR primer
caactacaacagccacaacgtct

SEQ ID NO: 10 - DNA, 23 bp, Artificial Sequence
  PCR primer
agttcaccttgatgccgttcttc

SEQ ID NO: 11 - DNA, 23 bp, Artificial Sequence
  PCR primer
caatgaatacggctacagcaaca

SEQ ID NO: 12 - DNA, 23 bp, Artificial Sequence
  PCR primer
gtggtccagggtttcttactcct

SEQ ID NO: 13 - DNA, 21 bp, Artificial Sequence
  PCR primer
ggcgagaaaccttaccactgt

SEQ ID NO: 14 - DNA, 22 bp, Artificial Sequence
  PCR primer
tactgaactctctctcctggca

SEQ ID NO: 15 - DNA, 20 bp, Artificial Sequence
  PCR primer
tcaagcagacgagcacaagc

SEQ ID NO: 16 - DNA, 20 bp, Artificial Sequence
  PCR primer
tacagtcccaaagccccagc

SEQ ID NO: 17 - DNA, 20 bp, Artificial Sequence
  PCR primer
ggaaacgtcatggacctgat

SEQ ID NO: 18 - DNA, 20 bp, Artificial Sequence
  PCR primer
ggaaggtgtttgtgcgtctt

SEQ ID NO: 19 - DNA, 17 bp, Artificial Sequence
  Antisense Linker 1
tagtcccttaagcggag

SEQ ID NO: 20 - DNA, 36 bp, Artificial Sequence
  Sense Linker 1
gtaatacgactcactatagggctccgcttaagggac

SEQ ID NO: 21 - DNA, 22 bp, Artificial Sequence
  Linker primer 1
gtaatacgactcactatagggc

SEQ ID NO: 22 - DNA, 22 bp, Artificial Sequence
  attB-F2 sequence
atgtaggtcacggtctcgaagc

SEQ ID NO: 23 - DNA, 21 bp, Artificial Sequence
  attB-R1
tcccgtgctcaccgtgaccac

SEQ ID NO: 24 - DNA, 19 bp, Artificial Sequence
  Linker primer 2
agggctccgcttaagggac

SEQ ID NO: 25 - DNA, 15 bp, Artificial Sequence
  attB-F3
cgaagccgcggtgcg

SEQ ID NO: 26 - DNA, 17 bp, Artificial Sequence
  attB-R2
actaccgccacctcgac

(19) United States
(12) Patent Application Publication
(10) Pub. No.: US 2012/0141441 A1
(43) Pub. Date: Jun. 7, 2012

(54) Methods and Compositions for Treatment of Muscular Dystrophy

(75) Inventors: MICHELE P. CALOS, MENLO PARK, CA (US); MARISA KAROW, Menlo Park, CA (US); CHRISTOPHER L. CHAVEZ, Santa Cruz, CA (US); Alfonso P. FARRUGGIO, MCALLEN, TX (US); CHUNLI ZHAO, PALO ALTO, CA (US); HASSAN CHAIB, STANFORD, CA (US); CHRISTOPHER BJORNSON, Menlo Park, CA (US); TAWNY NEAL, Menlo Park, CA (US); JONATHAN M. GEISINGER, Palo Alto, CA (US)

(73) Assignee: THE BOARD OF TRUSTEES OF THE LELAND STANFORD JUNIOR UNIVERSITY

(21) Appl. No.: 13/310,635

(22) Filed: Dec. 2, 2011

Related U.S. Application Data
(60) Provisional application No. 61/419,368, filed on Dec. 3, 2010.

(51) Int. Cl. A61K 48/00; A61P 21/00; C12N 15/85

(52) U.S. Cl. 424/93.21; 435/462

(57) ABSTRACT

The present disclosure provides methods for introducing a gene encoding a muscle membrane protein into a cell isolated from a subject to generate a genetically modified cell. The genetically modified cell may be introduced back, e.g., engrafted into the subject. The isolated cell may be additionally modified by introducing into the isolated cell a gene encoding one or more reprogramming transcription factors that induce the cell to form an induced pluripotent stem cell. The genetically modified cell may be differentiated in vitro to form muscle cell precursors before engrafting into the subject. Also provided are compositions comprising autologous cells isolated from a subject which cells comprise a muscle membrane protein gene integrated into a genome attachment site in the genome of the cell. The autologous cell may be an induced pluripotent cell or a mesenchymal stem cell, such as an adipose-derived mesenchymal stem cell (AD-MSC).

### CROSS-REFERENCE TO RELATED APPLICATIONS

This application claims the benefit of U.S. Provisional Application No. 61/419,368, filed on Dec. 3, 2010, which is herein incorporated by reference in its entirety.

### BACKGROUND

Muscular dystrophy generally refers to a disease or condition involving a deficiency of one or more muscle membrane proteins. For example, Duchenne muscular dystrophy (DMD) is a lethal X-linked genetic disorder caused by deficient dystrophin production. Mutations in the dystrophin gene, such as duplications/deletions of its exons appear to be the underlying defect. Dystrophin is a critical component of the dystrophin glycoprotein complex (DGC), which is involved in stabilizing interactions between the sarcolemma, the cytoskeleton, and the extracellular matrix of skeletal and cardiac muscles. A consequence of the DGC inefficiency is the enhanced rate of myofiber death during muscle contraction. Although satellite cells compensate for muscle fiber loss in the early stages of disease, eventually these progenitors become exhausted as evidenced by shorter telomere length and inability to generate new muscle. Subsequently fibrous and fatty connective tissues overtake the myofibers. Inflammatory cell infiltration, cytokine production, and complement activation are frequently observed. At the clinical level, these changes culminate in progressive muscle wasting, with the majority of patients becoming wheelchair-bound in their early teens. Patients generally succumb to cardiac/respiratory failure in their twenties, although rare cases of survival into the thirties have been reported.

With exception of corticosteroids, which have limited activity and carry numerous adverse effects, therapeutic interventions in muscular dystrophy have had limited, if any success. Current areas of investigation include replacement gene therapy, induction of exon-skipping by antisense to correct open reading frame mutations, and transfer of myoblast or other putative progenitor cells.

### SUMMARY

The present disclosure provides methods for introducing a gene encoding a muscle membrane protein into a cell isolated from a subject to generate a genetically modified cell. The genetically modified cell may be introduced back, e.g., engrafted into the subject. The isolated cell may be additionally modified by introducing into the isolated cell a gene encoding one or more reprogramming transcription factors that induce the cell to form an induced pluripotent stem cell. The genetically modified cell may be differentiated in vitro to form muscle cell precursors before engrafting into the subject. Also provided are compositions comprising autologous cells isolated from a subject which cells comprise a muscle membrane protein gene integrated into a genome attachment site in the genome of the cell. The autologous cell may be an induced pluripotent cell or a mesenchymal stem cell, such as an adipose-derived mesenchymal stem cell (AD-MSC).

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 provides a schematic of φC31-mediated integration of dystrophin gene in mammalian cells.

FIG. 2 shows a schematic of the therapeutic strategy using φC31 integrase and adipose derived mesenchymal stem cells (AD-MSC).

FIG. 3 illustrates the procedure for isolation and culture of AD-MSC.

FIG. 4 depicts analysis of AD-MSC surface marker expression.

FIG. 5 illustrates differentiation of AD-MSCs.

FIG. 6 shows analysis of nucleofected AD-MSCs by flow cytometry.

FIG. 7 illustrates the three-step reprogramming of MSCs to form iPSCs and genetic correction by addition of dystrophin.

FIG. 8 depicts a schematic of φC31-mediated integration of a plasmid carrying reprogramming transgenes into a pseudo attP site in a mammalian genome.

FIG. 9 shows a diagram of the p4FLR vector carrying four transcription factors separated by 2A sequences and driven by a CAG promoter.

FIG. 10 shows a close-up of recombinase sites from p4FLR, after genomic integration of reprogramming cassette.

FIG. 11 (panels A-C) shows engraftment in mice using luciferase live imaging.

FIG. 12 shows a diagram of the pTOBL5 vector.

FIG. 13 shows a diagram of the pKHLB_luc vector.

FIG. 14 shows an overview of generation of induced pluripotent stem cells (iPSCs) and removal of reprogramming factors using site-specific recombinases. (Panel A): Schematic overview of the recombinase strategy Mar. 29, 2011. (1): φC31 integrase is used to integrate the p4FLR plasmid at a preferred location and reprogram somatic cells. A clone with a single copy of the plasmid integrated at a safe, intergenic location is chosen. (2): Cre recombinase mediates precise excision of the reprogramming cassette. (Panel B): SSEA1 staining of an adipose-derived mesenchymal stem cell (ASC)-iPSC on day 20 after nucleofetion before picking (upper panel) and morphology of established iPSC lines generated from mouse embryonic fibroblast (MEF) and ASC starting populations shown by alkaline phosphatase staining compared with embryonic stem cells (lower panel). Scale bar=50 μm. (Panel C): Southern blot analysis of representative MEF-iPSC and ASC-iPSC lines before and after Cre-mediated excision of the reprogramming cassette, by using an EGFP probe. Both clones carried a single integration of the reprogramming cassette, which was no longer detectable after excision. (Panel D): Verification of the genomic integration site determined previously by linker-mediated polymerase chain reaction (PCR) using pairs of the respective genomic and plasmid-binding primers for both of the iPSC clones as depicted schematically (right panel). Genomic DNA of the parental cells was used as a negative control, proving specific product amplification (left panel). Cre-mediated excision of the reprogramming cassette was verified by amplification of the genomic integration locus (left panel). Genomic DNA of cells bearing the entire cassette served as negative controls, as in those samples the small PCR product could not be detected by using a combination of primers binding adjacent to the genomic integration site and within the recombined sites (dashed lines in right panel). PCR to test for pVI plasmid (lower left panel) showing the absence of the plasmid in established iPSC lines. Abbreviations: ASC, adipose-derived mesenchymal stem cell; Ctrl, control; ESC, embryonic stem cell; GFP, green fluorescent protein; iPSC, induced pluripotent stem cell; MEF, mouse embryonic fibroblast.

FIG. 15 shows pluripotency of induced pluripotent stem cells (iPSCs) before and after Cre-mediated excision. (Panel A): Quantitative real time-polymerase chain reaction data showing expression of Klf4, cMyc, GFP, Oct4, Sox2, Rex1, and Nanog in mouse embryonic fibroblast (MEF)-iPSC and adiposederived mesenchymal stem cell (ASC)-iPSC before and after removal of the reprogramming cassette as well as in the parental MEF and ASC and in embryonic stem cell controls. (Panel B): Promoter methylation status of Oct4 (left panel) and Nanog (right panel) in iPSC and iPSC-X lines. Five different CpG islands for each line were analyzed, indicated by their distance to the respective transcription start site. Open circles reflect low methylation (0%-25%), whereas gray circles represent medium (26%-75%) and black circles high (76%-100%) methylation. (Panel C). Immunofluorescence staining of Oct4, SSEA1, and EGFP in MEF-iPSC and ASC-iPSC before and after Cre recombinase treatment. Scale bar=50 μm. Abbreviations: ASC, adipose-derived mesenchymal stem cell; DAPI, 40,6-diamidino-2-phenylindole, dihydrochloride; ESC, embryonic stem cell; EGFP, enhanced green fluorescent protein; GFP, green fluorescent protein; iPSC, induced pluripotent stem cell; MEF, mouse embryonic fibroblast.

FIG. 16 shows in vivo studies of pluripotency of induced pluripotent stem cells (iPSCs) before and after Cre-mediated excision. (Panel A): Day 14 embryoid bodies were stained with the antibodies anti-smooth muscle actin, anti-Tuj 1, or anti-a-fetoprotein to show mesodermal, ectodermal, and endodermal differentiation in vitro, respectively. Alexa 594-labeled secondary antibodies were used, while Hoechst staining was used to visualize the nuclei. Figure represents merged pictures. Scale bar=50 μm. (Panel B): Histological samples obtained 4 weeks after injection of mouse embryonic fibroblast (MEF)-iPSC (left panel) or adipose-derived mesenchymal stem cell-iPSC (right panel) into SCID beige mice showing teratomas composed of cells with mesoderm, ectoderm, and endoderm lineages. (Panel C): Chimeric pups obtained after injection of MEF-iPSC (left image) and MEF-iPSC-X (right image) into blastocysts of albino B6 mice. iPSC gave rise to black fur. Abbreviations: AFP, anti-a-fetoprotein; ASC, adipose-derived mesenchymal stem cell; iPSC, induced pluripotent stem cell; MEF, mouse embryonic fibroblast; SCID, severe combined immunodeficiency; SMA, smooth muscle actin.

FIG. 17 shows verification of ASC origin. (Panel A) Flow cytometry plots showing the expression of Sca-1, CD29, and CD34 on ASC. All antibodies used, including the isotype control, were directly conjugated with FITC. (Panel B) ASC two weeks after being differentiated into the osteogenic lineage and stained with Alizarin Red (left panel) and into the adipogenic lineage shown by Oil Red 0 staining (right panel). Scale bars represents 50 μm.

FIG. 18 shows nucleofection efficiency by flow cytometry. MEF and ASC were nucleofected with p4FLR and pVI and subjected to flow cytometry 48 hours after nucleofection. The percentage of GFP-positive cells is shown in the right upper corner of each plot. nt=non transfected.

FIG. 19 shows Southern blot analysis of MEF- and ASC-iPSC clones. Examples of MEF- and ASC-clones analyzed by Southern blot. p4FLR plasmid, as well as ES cells from GFP transgenic mice, were used as controls for the GFP probe. Genomic DNA samples were digested with HindIII.

FIG. 20 shows chromosome counts and representative chromosome spreads from MEF- and ASC-iPSC, as well as ES cells. Scale bars represent 25 μm.

FIG. 21 shows bisulfite pyrosequencing of Oct4 and Nanog promoters. Quantitative methylation status of Oct4 promoter (top panel) and Nanog promoter (bottom panel) in iPSC and iPSC-X lines and the respective controls. Five different CpG sites as indicated in the legend were subjected to bisulfite pyrosequencing. Methylation status is shown as a percent.

FIG. 22 shows protein expression of Nanog and Sox2 in MEF- and ASC-iPSC before and after excision. Scale bars represent 50 μm.

FIG. 23 is a table showing primers used for qRT-PCR.

FIG. 24 is a table showing an overview of the 14 integration events analyzed by LM-PCR and sequencing.

FIG. 25 is a table summarizing φC31-mediated integration into the human genome.

### DETAILED DESCRIPTION OF THE INVENTION

The present disclosure provides compositions, pharmaceutical preparations, and methods that may generally be used to introduce a gene (e.g., a dystrophin gene or a dysferlin gene) into a cell isolated from a subject to generate a genetically modified cell. Also provided are kits for practicing the subject methods of the invention.

Before the present invention described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “a transcription factor” includes reference to one or more transcription factors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

“Recombinases” are a family of enzymes that mediate site-specific recombination between specific DNA sequences recognized by the recombinase.

“Altered recombinases” and “mutant recombinases” are used interchangeably herein to refer to recombinase enzymes in which the native, wild-type recombinase gene found in the organism of origin has been mutated in one or more positions relative to a parent recombinase (e.g., in one or more nucleotides, which may result in alterations of one or more amino acids in the altered recombinase relative to a parent recombinase). “Parent recombinase” is used to refer to the nucleotide and/or amino acid sequence of the recombinase from which the altered recombinase is generated. The parent recombinase can be a naturally occurring enzyme (i.e., a native or wild-type enzyme) or a non-naturally occurring enzyme (e.g., a genetically engineered enzyme). Altered recombinases exhibit a DNA binding specificity and/or level of activity that differs from that of the wild-type enzyme or other parent enzyme. Such altered binding specificity permits the recombinase to react with a given DNA sequence differently than would the parent enzyme, while an altered level of activity permits the recombinase to carry out the reaction at greater or lesser efficiency. A recombinase reaction typically includes binding to the recognition sequence and performing concerted cutting and ligation, resulting in strand exchanges between two recombining recognition sites.

A “unidirectional recombinase” is a recombinase that mediates irreversible site-specific recombination between specific DNA sequences recognized by the recombinase. Examples of unidirectional recombinase include integrases such as the φC31 integrase, R4 integrase, a mutated integrase, such as a mutated φC31 integrase, mutated R4 integrase that retains unidirectional, site-specific recombination activity, or a bi-directional recombinase modified so as to be unidirectional, such as a Cre recombinase that has been modified to become unidirectional. The native attB and attP recognition sites of phage φC31 (i.e., bacteriophage phiC31) are generally about 34 to 40 nucleotides in length (Groth et al. Proc Natl Acad Sci USA 97:5995-6000 (2000)). These sites are typically arranged as follows: attB comprises a first DNA sequence attB5′, a core region, and a second DNA sequence attB3′, in the relative order from 5′ to 3′: attB5′-core region-attB3′. attP comprises a first DNA sequence attP5′, a core region, and a second DNA sequence attP3′, in the relative order from 5′ to 3′: attP5′-core region-attP3′. The core region of attP and attB specific for φC31 has the sequence 5′-TTG-3′. The full-length, native attP and attB sequences for φC31 integrase are provided in US PG Pub No. 20020094516, which is incorporated herein by reference. Other unidirectional recombinases include R4 phage integrase. The full-length, native attB and attP sequences for the R4 phage integrase are also provided in US PG Pub No. 20020094516. Other unidirectional recombinases include Bxb1 recombinase, U153 recombinase, and TP901 recombinase. cDNA sequences encoding these recombinases are provided in WO/2006/026537, which is herein incorporated by reference. Action of the integrase upon the recognition sites recognized by the integrase is unidirectional in that the enzymatic reaction produces nucleic acid recombination products that are not effective substrates of the integrase. This results in stable integration with little or no detectable recombinase-mediated excision, i.e., recombination that is irreversible. The recombination product of integrase action upon the recognition site pair comprises, for example, in order from 5′ to 3′: attB5′-recombination product site sequence-attP3′, and attP5′-recombination product site sequence-attB3′. Thus, where the targeting vector comprises an attB site as the vector attachment site and the target site in the genome comprises an attP sequence, a typical recombination product comprises the sequence (from 5′ to 3′): attP5′-TTG-attB3′ {targeting vector sequence}attB5′-TTG-attP3′.

A “recognition site” is a DNA sequence that serves a substrate for a wild-type or altered recombinase so as to provide for site-specific recombination. In general, the unidirectional recombinases used herein involve two recognition sites, one that is positioned in the integration site (the site into which a nucleic acid is to be integrated) and another adjacent a nucleic acid of interest to be introduced into the integration site. As used herein the phrase “target site in a genome”, “genome attachment site”, or “genome target site” refer to a recognition site for a unidirectional recombinase, which target site is positioned in the integration site in the genome of the host cell. As used herein the phrase “vector attachment site” thereof refer to a recognition site for a unidirectional recombinase, which vector attachment site is present in a targeting vector adjacent to a nucleic acid of interest to be introduced into a target site in the genome. For example, the recognition sites for phage integrases are generically referred to as attB, which is present in the bacterial genome (into which nucleic acid is to be inserted) and attP (which is present in the phage nucleic acid adjacent the nucleic acid for insertion into the bacterial genome). Recognition sites can be native (endogenous to a phage) or altered relative to a native sequence.

Use of the term “recognize” in the context of a recombinase “recognizes” a recognition sequence, is meant to refer to the ability of the recombinase to interact with the recognition site and facilitate site-specific recombination.

A recognition site “native” or “endogenous” to the genome, as used herein, means a recognition site that occurs naturally in the genome of a cell (i.e., the sites are not introduced into the genome, for example, by recombinant means). A recognition site endogenous or native to a genome is also referred to herein as “endogenous target site”. A recognition site that has been introduced into the genome of a cell, for example, by recombinant means is referred to herein as “a target site” or “a non-endogenous target site”.

A “pseudo-site” is a DNA sequence comprising a recognition site that is recognized by a recombinase enzyme where the recognition site differs in one or more nucleotides from a wild-type recombinase recognition sequence and/or is present as an endogenous sequence in a genome that differs from the sequence of a genome where the wild-type recognition sequence for the recombinase resides. In some embodiments a “pseudo attP site” or “pseudo attB site” refer to pseudo sites that are similar to the recognitions site for wild-type phage (attP) or bacterial (attB) attachment site sequences, respectively, for phage integrase enzymes, such as the phage φC31. In many of the embodiments disclosed herein, the pseudo attP site is present in the genome of a host cell, while the attB site or the pseudo attB site is present on a targeting vector. “Pseudo att site” is a more general term that can refer to either a pseudo attP site or a pseudo attB site. It is understood that att sites or pseudo att sites may be present on linear, circular, or supercoiled nucleic acid molecules.

A “bidirectional recombinase” refers to a recombinase that, upon binding to compatible targeting sites, produces nucleic acid recombination products that are effective substrates for the recombinase. For example, when a bidirectional recombinase mediates recombination between two compatible targeting sites that flank a nucleic acid sequence and are arranged in the same orientation, e.g., head-to-head or tail-to-tail, the recombination between the two compatible targeting sites yields a single targeting site which can be used as a substrate for the recombinase. Well-known examples of bidirectional recombinases can be found in Cre-lox, FLP/FRT, R/RS, Gin/gix, and pSR1 system (e.g. N. L. Craig, Annu. Rev. Genet. 22:17, 1988; Odell et al., Homologous Recomb. Gene Silencing Plants, 1994, pp. 219-270, Paszkowski, Jerzy, ed. Kluwer: Dordrecht, Germany).

A “vector” is capable of transferring gene sequences to host cells. Typically, “vector construct,” “expression vector,” and “gene transfer vector,” mean any nucleic acid construct capable of directing the expression of a gene of interest and which can transfer gene sequences to host cells. Thus, the term includes cloning and expression vehicles, as well as integrating vectors. The vector may be present in a supercoiled form, a circular form, a linear form, or as a mixture of two or more of these forms.

An “expression cassette” comprises any nucleic acid construct capable of directing the expression of a gene/coding sequence of interest. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

A first polynucleotide is “derived from” a second polynucleotide if it has the same or substantially the same nucleotide sequence as a region of the second polynucleotide, its cDNA, or complements thereof.

A first polypeptide is “derived from” a second polypeptide if it is (i) encoded by a first polynucleotide derived from a second polynucleotide, or (ii) displays sequence identity to the second polypeptides as described above.

When a recombinase is “derived from a phage” the recombinase need not be explicitly produced by the phage itself, the phage is simply considered to be the original source of the recombinase and coding sequences thereof. Recombinases can, for example, be produced recombinantly or synthetically, by methods known in the art, or alternatively, recombinases may be purified from phage infected bacterial cultures.

By “ex vivo” it is meant that cells or organs are modified outside of the body. Such cells or organs are typically returned to a living body.

The “host cell” or “the cell” as used herein can be any cell isolated from a subject (autologous cell) which cell is amenable to genetic modification using the methods disclosed herein. Exemplary host cells include, but are not necessarily limited to somatic cells (e.g., muscle, bone, cartilage, ligament, tendon, skin (dermis, epidermis, and the like), cells of the viscera (e.g., lung, liver, pancreas, gastrointestinal tract (mouth, stomach, intestine), and the like), stem cells (e.g., embryonic stem cells (e.g., cells having an embryonic stem cell phenotype, adult stem cells, pluripotent stem cells, hematopoietic stem cells, mesenchymal stem cells, and the like), germ cells (e.g., primordial germ cells, embryonic germ cells, and the like).

As used herein the term “isolated” is meant to describe cell of interest (e.g., a stem cell) that is in an environment different from that in which the element naturally occurs.

“Purified” as used herein refers to a cell removed from an environment in which it was produced and is at least 60% free, preferably 75% free, and most preferably 90% free from other components with which it is naturally associated or with which it was otherwise associated with during production.

The phrases “operably associated” and “operably linked” refer to functionally related nucleic acid sequences. By way of example, a regulatory sequence is operably linked or operably associated with a protein encoding nucleic acid sequence if the regulatory sequence can exert an effect on the expression of the encoded protein. In another example, a promoter is operably linked or operably associated with a protein encoding nucleic acid sequence if the promoter controls the transcription of the encoded protein. While operably associated or operably linked nucleic acid sequences can be contiguous with the nucleic acid sequence that they control, the phrases “operably associated” and “operably linked” are not meant to be limited to those situations in which the regulatory sequences are contiguous with the nucleic acid sequences they control.

The term “gene” as used herein includes sequences of nucleic acids that when present in an appropriate host cell facilitates production of a gene product. “Genes” can include nucleic acid sequences that encode proteins, and sequences that do not encode proteins, and includes genes that are endogenous to a host cell or are completely or partially recombinant (e.g., due to introduction of an exogenous polynucleotide encoding a promoter and a coding sequence, or introduction of a heterologous promoter adjacent an endogenous coding sequence, into a host cell). For example, the term “gene” includes nucleic acid that can be composed of exons and introns. Sequences that code for proteins are, for example, sequences that are contained within exons in an open reading frame between a start codon and a stop codon. “Gene” as used herein refers to a nucleic acid that includes, for example, regulatory sequences such as promoters, enhancers and all other sequences known in the art that control the transcription, expression, or activity of a nucleic acid sequence operably linked or operably associated to the regulatory sequence, whether the nucleic acid sequence comprises coding sequences or non-coding sequences. In one context, for example, “gene” may be used to describe a nucleic acid comprising regulatory sequences such as promoter or enhancer and coding and non-coding sequences. The expression of a recombinant gene may be controlled by one or more heterologous regulatory sequences. “Heterologous” refers to two elements that are not normally associated in nature.

“Regulatory elements” are nucleic acid sequences that regulate, induce, repress, or otherwise mediate the transcription, translation of a protein or RNA coded by a nucleic acid sequence with which they are operably linked or operably associated. Typically, a regulatory element or sequence such as, for example, an enhancer or repressor sequence, is operably linked or operably associated with a protein coding nucleic acid sequence if the regulatory element or regulatory sequence mediates the level of transcription, translation or expression of the protein coding nucleic acid sequence in response to the presence or absence of one or more regulatory factors that control transcription, translation and/or expression. Regulatory factors include, for example, transcription factors. Regulatory sequences may be found in introns.

Regulatory sequences or elements include, for example, “TATAA” boxes, “CAAT” boxes, differentiation-specific elements, cAMP binding protein response elements, sterol regulatory elements, serum response elements, glucocorticoid response elements, transcription factor binding elements such as, for example, SPI binding elements, and the like. A “CAAT” box is typically located upstream (in the 5′ direction) from the start codon of a eukaryotic nucleic acid sequence encoding a protein or RNA. Examples of other regulatory sequences include splicing signals, polyadenylation signals, termination signals, and the like. Numerous regulatory sequences are known in the art.

The term “enhancer” and phrase “enhancer sequence” refer to a variety of regulatory sequences that can increase the efficiency of transcription, without regard to the orientation of the enhancer sequence or its distance or position in space from the promoter, transcription start site, or first codon of the nucleic acid sequence encoding a protein with which the enhancer is operably linked or associated.

The term “promoter” refers to a nucleic acid sequence that does not code for a protein, and that is operably linked or operably associated to a protein coding or RNA coding nucleic acid sequence such that the transcription of the operably linked or operably associated protein coding or RNA coding nucleic acid sequence is controlled by the promoter. Typically, eukaryotic promoters comprise between 100 and 5,000 base pairs, although this length range is not meant to be limiting with respect to the term “promoter” as used herein. Although typically found 5′ to the protein coding nucleic acid sequence to which they are operably linked or operably associated, promoters can be found in intron sequences as well. The term “promoter” is meant to include regulatory sequences operably linked or operably associated with the same protein or RNA encoding sequence that is operably linked or operably associated with the promoter. Promoters can comprise many elements, including regulatory elements. The term “promoter” comprises promoters that are inducible, wherein the transcription of the operably linked nucleic acid sequence encoding the protein is increased in response to an inducing agent. The term “promoter” may also comprise promoters that are constitutive, or not regulated by an inducing agent.

“Nucleotide analogs” include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil, and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

The phrase “nuclear uptake enhancing modification” refers to a modification of a naturally occurring or non-naturally occurring polynucleotide that provides for enhanced nuclear uptake of the modified polynucleotide. An example of a “nuclear uptake enhancing modification” is a stabilizing modification, such as a modified inter-nucleotide linkage, that confers sufficient stability on a molecule, such as a nucleic acid, to render it sufficiently resistant to degradation (e.g., by nucleases) such that the associated nucleic acid can accumulate in the nucleus of a cell when exogenously introduced into the cell. In this example, entry into the cell's nucleus is facilitated by the ability of the modified nucleic acid to resist nucleases sufficiently well such that an effective concentration of the nucleic acid can be achieved inside the nucleus. An effective concentration is a concentration that results in a detectable change in the transcription or activity of a gene or target sequence as the result of the accumulation of nucleic acid within the nucleus.

The phrase “pharmaceutically acceptable carrier” refers to compositions that facilitate the introduction of a polynucleotide into a cell and includes but is not limited to solvents or dispersants, coatings, anti-infective agents, isotonic agents, agents that mediate absorption time or release of the polynucleotide. Examples of “pharmaceutically acceptable carriers” include liposomes that can be neutral or cationic, can also comprise molecules such as chloroquine and 1,2-dioleoyl-sn-glycero-3-phosphatidyle-thanolamine, which can help destabilize endosomes and thereby aid in delivery of liposome contents into a cell, including a cell's nucleus. Examples of other pharmaceutically acceptable carriers include poly-L-lysine, polyalkylcyanoacrylate nanoparticles, polyethyleneimines, and any suitable PAMAM dendrimers (polyamidoamine) known in the art with various cores such as, for example, ethylenediamine cores, and various surface functional groups such as, for example, cationic and anionic functional groups, amines, ethanolamines, aminodecyl.

The term “pluripotent cell” as used herein refers to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Pluripotent stem cells can give rise to any fetal or adult cell type. Induced pluripotent stem cells are a type of pluripotent stem cells.

The term “multipotent cell” as used herein refers to a cell that has potential to give rise to cells from multiple, but a limited number of lineages.

As used herein, the term “stem cell” refers to an undifferentiated cell that can be induced to proliferate. The stem cell is capable of self-maintenance, meaning that with each cell division, one daughter cell will also be a stem cell. Stem cells can be obtained from embryonic, fetal, post-natal, juvenile or adult tissue.

The term “progenitor cell”, as used herein, refers to an undifferentiated cell derived from a stem cell, and is not itself a stem cell. Some progenitor cells can produce progeny that are capable of differentiating into more than one cell type.

The term “induced pluripotent stem cell” (or “iPS cell” or “iPSC”), as used herein, refers to a stem cell induced from a non-pluripotent cell, e.g., a multipotent cell (for example, mesenchymal stem cell, adult stem cell, hematopoietic cell), a somatic cell (For example, a differentiated somatic cell, e.g., fibroblast), and that has a higher potency than the non-pluripotent cell. iPS cells are capable of self-renewal and differentiation into mature cells. iPS may also be capable of differentiation into progenitor cells that can produce progeny that are capable of differentiating into more than one cell type.

The term “treating” or “treatment” of a condition or disease includes providing a clinical benefit to a subject, and includes: (1) inhibiting the disease, i.e., arresting or reducing the development of the disease or its symptoms, or (2) relieving the disease, i.e., causing regression of the disease or its clinical symptoms.

The terms “individual,” “subject,” “host,” and “patient,” are used interchangeably herein and refer to any mammalian subject for whom treatment or therapy is desired, particularly humans. The mammalian subject may be canine, equine, bovine, or human.

### OVERVIEW

The present disclosure provides methods for introducing a gene encoding a muscle membrane protein into a cell isolated from a subject to generate a genetically modified cell. The genetically modified cell may be introduced back, e.g., engrafted into the subject. The isolated cell may be additionally modified by introducing into the isolated cell one or more genes encoding reprogramming transcription factors that induce the cell to form an induced pluripotent stem cell. The genetically modified cell may be differentiated in vitro to form muscle cell precursors before engrafting into the subject. Also provided is a composition comprising autologous cells isolated from a subject, wherein the cells comprise a gene encoding a muscle membrane protein integrated into a genome attachment site in the genome of the cell. The autologous cells may be induced pluripotent cells or mesenchymal stem cells, such as AD-MSCs.

### Methods

As noted above, a method for introducing a gene encoding a muscle membrane protein into a cell isolated from a subject to generate a genetically modified cell is provided. The isolated cell may be additionally modified by introducing into the isolated cell one or more genes encoding reprogramming transcription factors that induce the cell to form an induced pluripotent stem cell. The genetically modified cell may be differentiated in vitro to form muscle cell precursors before engrafting into the subject.

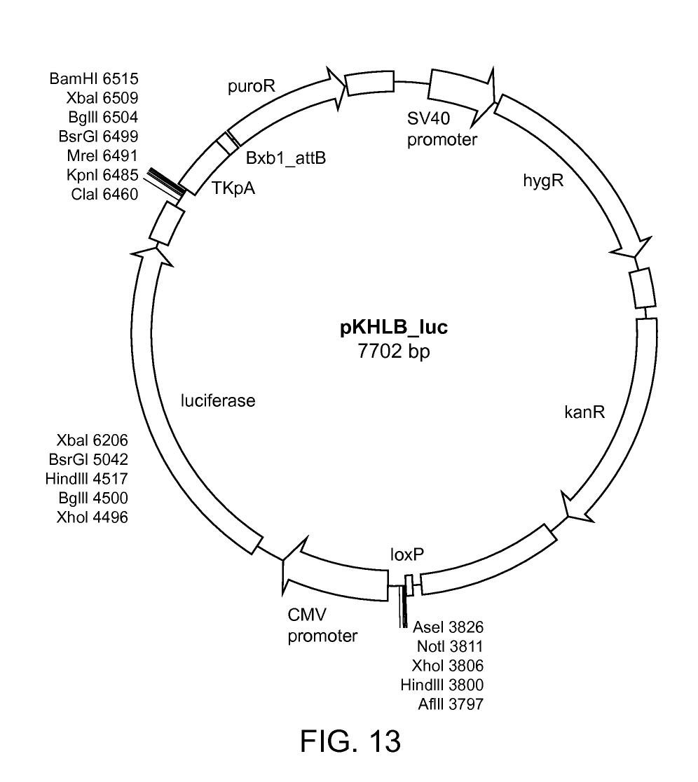

In certain embodiments, the method includes introducing into a cell isolated from a subject: (i) an expression cassette comprising a polynucleotide encoding a first site-specific unidirectional recombinase and (ii) a first targeting vector comprising a first vector attachment site recognized by the first site-specific unidirectional recombinase, a target site recognized by a second site-specific unidirectional recombinase, and a nucleic acid sequence encoding one or more reprogramming transcription factors, wherein the reprogramming transcription factors induce the cell to form a pluripotent stem cell; maintaining the cell under conditions sufficient for the first targeting vector to integrate into an endogenous target site in the genome of the cell by a recombination event between the first vector attachment site and the endogenous target site mediated by the first site-specific unidirectional recombinase and to induce the cell to form an induced pluripotent stem cell, wherein the induced pluripotent stem cell comprises the target site in the genome; introducing into the induced pluripotent stem cell: (i) an expression cassette comprising a polynucleotide encoding the second site-specific unidirectional recombinase and (ii) a second targeting vector comprising a second vector attachment site recognized by the second site-specific unidirectional recombinase and a nucleic acid encoding a muscle membrane protein; and maintaining the induced pluripotent stem cell under conditions sufficient for the second targeting vector to integrate into the target site in the genome of the induced pluripotent stem cell by a recombination event between the second vector attachment site and the target site mediated by the second site-specific unidirectional recombinase to produce an induced pluripotent stem cell comprising the gene encoding the muscle membrane protein. In some embodiments, the second targeting vector comprises one or more targeting sites that are specific for a bidirectional recombinase, e.g., Cre recombinase. An example of such a targeting vector is shown in FIG. 13.

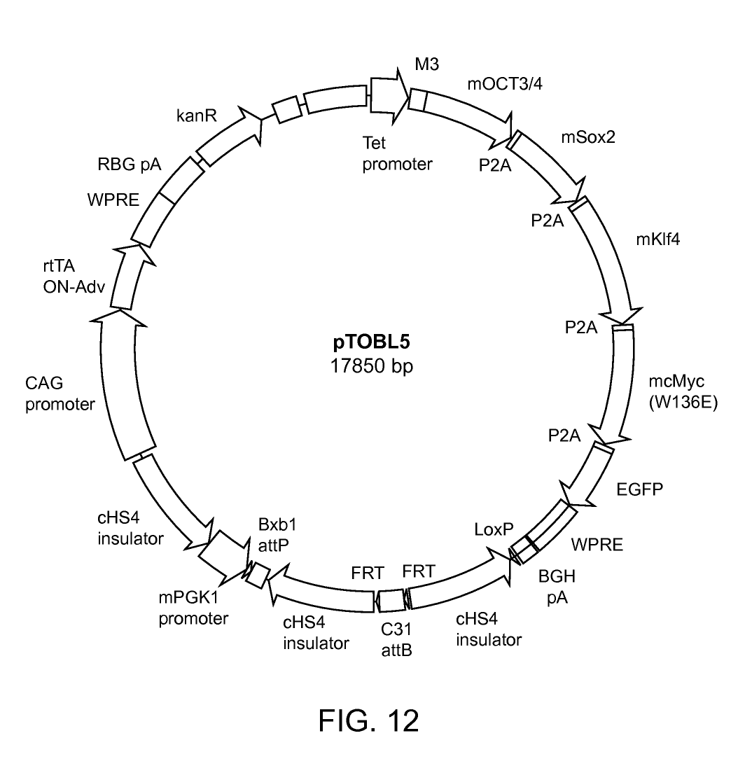

In certain embodiments, the nucleic acid encoding the one or more reprogramming transcription factors comprises one or more targeting sites that are specific for a bidirectional recombinase, e.g., Cre recombinase. An example of such a vector is the plasmid shown in FIG. 12. In some embodiments, the nucleic acid encoding the one or more reprogramming transcription factors is flanked by two compatible targeting sites that are specific for a bidirectional recombinase, wherein the two compatible targeting sites are arranged in the same orientation. In some embodiments, the method comprises excising the nucleic acid encoding the one or more reprogramming transcription factors from the induced pluripotent stem cell by exposing the induced pluripotent stem cell to the bidirectional recombinase, wherein the bidirectional recombinase mediates a recombination event between two compatible targeting sites flanking the one or more reprogramming transcription factor genes. In some embodiments, the method comprises excising portions of both the first and second targeting vectors from the induced pluripotent stem cell by exposing the induced pluripotent stem cell to the bidirectional recombinase, wherein the bidirectional recombinase mediates a recombination event between a first targeting site on the first targeting vector and a second compatible targeting site on the second targeting vector. The induced pluripotent stem cell may be exposed to the bidirectional recombinase by contacting the induced pluripotent stem cell with the bidirectional recombinase, for example, by incubating the induced pluripotent stem cell with the bidirectional recombinase by adding the bidirectional recombinase to a solution (e.g., a buffer, culture medium, and the like) in which the induced pluripotent stem cell is present. The induced pluripotent stem cell may be exposed to the bidirectional recombinase by introducing a nucleic acid encoding the bidirectional recombinase into the induced pluripotent stem cell.

In certain embodiments, the nucleic acid encoding the one or more reprogramming transcription factors is flanked by two compatible targeting sites specific for a unidirectional recombinase, wherein the two compatible targeting sites are arranged in the same orientation, and the method comprises excising the nucleic acid encoding the one or more reprogramming transcription factors from the induced pluripotent stem cell by exposing the induced pluripotent stem cell to a unidirectional recombinase, wherein the unidirectional recombinase mediates a recombination event between the two compatible targeting sites. In certain embodiments, the nucleic acid encoding the one or more reprogramming transcription factors (i.e. the first targeting vector) comprises one or more targeting sites that are specific for a unidirectional recombinase, and the second targeting vector comprises one or more targeting sites that specific for the same unidirectional recombinase, and the method comprises excising portions of both the first and second targeting vectors from the induced pluripotent stem cell by exposing the cell to the unidirectional recombinase, wherein the unidirectional recombinase mediates a recombination event between a first targeting site on the first targeting vector and a second compatible targeting site on the second targeting vector. The induced pluripotent stem cell may be exposed to the unidirectional recombinase in a manner as described above for exposing the induced pluripotent stem cell to a bidirectional recombinase.

Although, the above-described method includes introducing the one or more reprogramming transcription factors first, generating iPSCs, and then introducing the gene encoding a muscle membrane protein, the sequence of steps may be altered to introduce the muscle membrane-encoding gene first, followed by introduction of the one or more reprogramming genes, and generation of iPSCs.

In certain embodiments, the induced pluripotent stem cell comprising the muscle membrane-encoding gene may be introduced back into the subject from which the cell was isolated. Thus, the methods provided herein may be used to generate an autologous iPSCs that may be used for providing therapy to the subject.

In certain embodiments, the induced pluripotent stem cell comprising the muscle membrane-encoding gene may be differentiated in vitro to form muscles precursor cells before introducing back into the subject from which the cell was isolated. Thus, the methods provided herein may be used to introduce a muscle membrane-encoding gene into the subject.

In one embodiment, an integrase recognition site pair includes a target site present in the genome of a host cell and a vector attachment site present in a targeting vector as based on recognition sites that serve as substrates for a phage integrase, such as a φC31 integrase or R4 integrase. It is noted that in the native “φC31 system”, attB is present in the target bacterial genome and attP is normally present in the phage genome to facilitate integration of the phage genome into the bacterial host cell. However, the present disclosure provides a φC31 system in which an “attP” sequence (attP or attP pseudo-sequence or engineered sequence derived from attP) is present in the target genome and the “attB” sequence (attB or attB pseudo-sequence or engineered sequence derived from attB) is present in the targeting vector. In certain embodiments, however, the “attP” sequence may be present in the targeting vector and the “attB” sequence may be present in the target genome.

In certain embodiments, the first site-specific unidirectional recombinase may be an integrase, such as a φC31 integrase or a mutant thereof. The first vector attachment site present in the first targeting vector may be a recognition sequence, such as an attB sequence that is recognized by the φC31 integrase or a mutant thereof. The target site (present on the first targeting vector) recognized by a second site-specific unidirectional recombinase may be an R4 integrase specific attP site and the second site-specific unidirectional recombinase may be R4 integrase. Alternatively, the target site (present on the first targeting vector) recognized by a second site-specific unidirectional recombinase may be an attP site for Bxb1 recombinase or a mutant thereof and the second site-specific unidirectional recombinase may be a Bxb1 recombinase or a mutant thereof. In some embodiments, the first vector attachment site may be a recognition sequence that is recognized by phiC31 integrase or a mutant thereof, and the second vector attachment site may be a recognition sequence that is recognized by Bxb1 recombinase. Recognition sequences for Bxb1 reombinase are provided in WO/2006/026537. In certain embodiments, the first site-specific unidirectional recombinase a φC31 integrase or a mutant thereof and the endogenous target site in the genome of the cell may be a pseudo-attP site which is recognized by the φC31 integrase or a mutant thereof. In certain embodiments, the second vector attachment site recognized by the second site-specific unidirectional recombinase is a recognition site specific for R4 intergrase or a mutant thereof, for example an attB site specific for R4 integrase or a mutant thereof, or the second vector attachment is a recognition site for Bxb1 recombinase or a mutant thereof. Those of skill in the art will recognize that various combinations of first and second unidirectional recombination recognition sites may be used with any of the unidirectional recombinases disclosed herein to carry out the subject methods.

In certain cases, the first targeting vector may include a promoter sequence, one or more reprogramming transcription factors encoding nucleic acid sequences, an attB vector attachment site for an integrase, e.g., a φC31 integrase, and an attP site to serve as a genome target site or genome attachment site once the vector is integrated into the host genome. In certain embodiments, the first targeting vector may also comprise one or more targeting sites that are recognized by a second unidirectional recombinase, or that are recognized by a bidirectional recombinase. In some embodiments, the one or more reprogramming transcription factors encoding nucleic acid sequences may be flanked by two compatible targeting sites arranged in the same orientation, e.g., loxP sites. The second targeting vector may include an attB vector attachment site and a nucleic acid encoding a muscle membrane protein. In some embodiments, the second targeting vector may include one or more targeting sites that are recognized by a second unidirectional recombinase, or that are recognized by a bidirectional recombinase.

In certain embodiments, the method includes introducing a muscle membrane-encoding gene into a subject, the method comprising: introducing into an adipose-derived mesenchymal stem cell or a fibroblast cell isolated from the subject: (i) a first expression cassette comprising a polynucleotide encoding a first site-specific unidirectional recombinase and (ii) a first targeting vector comprising a nucleic acid encoding a muscle membrane-encoding gene and a first vector attachment site recognized by the first site-specific unidirectional recombinase; maintaining the adipose-derived mesenchymal stem cell or the fibroblast cell under conditions sufficient for the targeting vector to integrate into an endogenous target site in the genome of the cell by a recombination event between the first vector attachment site and the endogenous target site mediated by the first site-specific unidirectional recombinase to produce a genetically modified cell; and engrafting the genetically modified cell in the subject. The method may further comprise differentiating the genetically modified cell into a muscle precursor cell before the engrafting.

In certain embodiments, the first targeting vector may comprise a target site recognized by a second site-specific unidirectional recombinase and the target site recognized by a second site-specific unidirectional recombinase is present in the genome of the genetically modified stem cell, and the method comprises, before the engrafting step, introducing into the genetically modified stem cell: (iii) a second expression cassette encoding the second site-specific unidirectional recombinase and (iv) a second targeting vector comprising a second vector attachment site recognized by the second site-specific unidirectional recombinase and a nucleic acid sequence encoding one or more reprogramming transcription factors, wherein the reprogramming transcription factors induce the genetically modified cell to form a pluripotent stem cell; and maintaining the genetically modified cell under conditions sufficient for the second targeting vector to integrate into the target site present in the genome of the cell by a recombination event between the second vector attachment site and the target site mediated by the second site-specific unidirectional recombinase and to induce the genetically modified cell to form an induced pluripotent stem cell.

### Vector Attachment Site

The vector attachment site is a domain of nucleotides that serves as a substrate for the integrase with which it is employed, i.e., it recombines with a genome target site in an integrase mediated recombination event. The vector attachment site may vary in length, but typically ranges from about 20 to about 300 nt, usually from about 25 to about 200 nt, and more usually from about 30 to 40 nt. The vector attachment site has a sequence that is different from the genome attachment site, such that a recombination event mediated by the integrase is a unidirectional or “one-way” recombination event.

Exemplary vector attachment sites comprise a first DNA sequence attB5′, a core region, and a second DNA sequence attB3′, in the relative order from 5′ to 3′ attB5′-core region-attB3′.

In one embodiment, the vector attachment site is an attachment site for recognition by an integrase, sometimes a φC31 phage integrase or a mutant thereof, e.g., an attB site, or a pseudo-site sequence based on the attB site that contains at least one nucleotide difference from a wild-type attB site.

### Integrases

The unidirectional recombinase(s) useful in the methods described herein include wild-type or mutant recombinases, e.g., φC31 integrase, R4 integrase, Bxb1 integrase, TP901-1 integrase, A118 integrase, ΦFC1 integrase, and the like. A mutant integrase differs by at least one amino acid residue from a naturally-occurring or wild-type integrase.

Action of the integrase upon the recognition site pair of the vector attachment site and the genome target site or genome attachment site yields a recombination product that is not generally susceptible to recombination, e.g., recombination of the integrase recombination product by the integrase is insignificant or undetectable.

### Genome Target Site

The genome target site or genome attachment site is a target site that is a stretch, domain or region of nucleotides that is present in the host cell genome and is recognized by a unidirectional recombinase. The genome target site or genome attachment site is the desired integration site and is a region, site or domain of the host cell genome that serves as the integration point. The genome attachment site is a domain of nucleotides that serves as a substrate for a site-specific unidirectional recombinase, e.g., an integrase, and it recombines with a vector attachment site in an integrase-mediated recombination event. The genome attachment site may vary in length, but typically ranges from about 20 to about 300 nt, usually from about 23 to about 100 nt, more usually from about 28 to about 50 nt, and generally about 40 nt.

Exemplary genome attachment sites generally comprise a first DNA sequence attP5′, a core region, and a second DNA sequence attP3′, in the relative order from 5′ to 3′ attP5′-core region-attP3′. The recombination product of integrase action upon the genome attachment site and the vector attachment site comprises, for example, in order from 5′ to 3′ in the genome: attR-vector sequence-attL, where attR is the combination of the 5′ region of the attP site and 3′ region of the attB site and attL is a combination of 5′ region of the attB site and 3′ region of the attP site when attP is the genome attachment site and attB is the vector attachment site. In many embodiments, the genome attachment site is an attachment site for recognition by a phage integrase.

### Targeting Vector Production

The targeting vector employed in the subject methods is one that integrates the nucleic acid that it carries into the host cell genome at a site specific for a site-specific unidirectional recombinase.

Obtaining a targeting vector that provides for this requisite site-specific integration design includes identification of the type of attachment site present in the target genome, a vector attachment site; and a unidirectional site-specific recombinase for recognition of the target and vector attachment sites; and construction of a vector that incorporates the identified elements.

Targeting vectors may contain more than one vector attachment site, and may contain one or more additional recombination sites (e.g., lox sites, att sites, etc.) other than the vector attachment site.

In certain embodiments, the targeting vector useful in the methods disclosed herein may include a promoter sequence, one or more nucleic acid sequences encoding reprogramming transcription factors, an attB vector attachment site for an integrase, e.g., a φC31 integrase, and an attP site to serve as a genome target site or genome attachment site once the vector is integrated into the host genome. In some embodiments, a targeting vector may also comprise one or more additional recombination sites. Such additional recombination sites may be sites that are recognized by unidirectional, site-specific recombinases, or may be sites that are recognized by bidirectional recombinases. In some embodiments, the one or more nucleic acid sequences encoding reprogramming transcription factors may be flanked by two compatible targeting sites arranged in the same orientation, e.g., loxP sites.

In certain embodiments, the targeting vector useful in the methods disclosed herein may include a promoter sequence, a muscle membrane protein-encoding nucleic acid sequence, an attB vector attachment site for an integrase, e.g., R4 integrase, Bxb1 recombinase, etc.

In certain embodiments, the targeting vector useful in the methods disclosed herein may include a promoter sequence, a muscle membrane protein-encoding nucleic acid sequence, an attB vector attachment site for an integrase, e.g., a φC31 integrase, and an attP site to serve as a genome target site or genome attachment site once the vector is integrated into the host genome.

In certain embodiments, the targeting vector useful in the methods disclosed herein may include a promoter sequence, one or more reprogramming transcription factor-encoding nucleic acid sequences and an attB vector attachment site for an integrase, e.g., a R4 integrase or Bxb1 recombinase. In some embodiments, the one or more reprogramming transcription factor-encoding nucleic acid sequences may be flanked by two compatible targeting sites arranged in the same orientation, e.g., loxP sites.

Genes Encoding Muscle Membrane Proteins

The terms “muscle membrane protein-encoding gene” or “gene encoding a muscle membrane protein,” as used interchangeably herein, generally refer to cDNA derived from mRNA encoding a muscle membrane protein. The source of the mRNA depends upon the subject whose cells are being modified to introduce the muscle membrane protein-encoding gene. In general, the when the subject is human, human mRNA is used. Examples of such proteins include, but are not limited to, dystrophin, dysferlin, and the like.

Dystrophin

In certain embodiments, the muscle membrane protein-encoding gene is a dystrophin gene. In some embodiments, this gene is a cDNA derived from dystrophin transcript variant Dp427c mRNA of Genbank Accession No. NM_{—}000109 (encoding dystrophin of Accession No. NP_{—}000100), or dystrophin transcript variant Dp427m mRNA of Accession No. NM_{—}004006 (encoding dystrophin of Accession No. NP_{—}003997), or dystrophin transcript variant Dp4271 mRNA of Accession No. NM_{—}004007 (encoding dystrophin of Accession No. NP_{—}003998), or dystrophin transcript variant Dp427 μl mRNA of Accession No. NM_{—}004009 (encoding dystrophin of Accession No. NP_{—}004000). In certain embodiments, the term “dystrophin gene” refers to a nucleic acid sequence encoding a dystrophin protein of Accession No. NP_{—}003997.1.

In certain embodiments, the dystrophin gene may be a “dystrophin minigene”. The term refers to dystrophin constructs created by extensive deletions in the central rod domain plus extensive deletions in the C-terminal domain of the human dystrophin cDNA. In addition, the dystrophin minigenes may contain a modified N-terminal domain in which DNA sequences surrounding the original protein translation initiation codon ATG are modified. The modified sequences enhance the mini-dystrophin protein synthesis. Alternatively, the dystrophin minigene may be a hybrid gene in which some of the domains are substituted with homologous domains from utrophin or spectrin genes (Tinsley et al, Nature 360, 591-593, 1992; Koenig et al. Cell 53, 219-216, 1988). For example, the N-terminal and/or the C-terminal domains of dystrophin may be substituted with the utrophin counterparts in the dystrophin minigenes. Similarly, the central rod domain may consist of rod repeats from utrophin or spectrin genes. The term “mini-dystrophin” refers to the polypeptides encoded by the dystrophin minigenes. Dystrophin minigenes are described in U.S. Pat. Nos. 7,510,867 and 7,001,761, which are hereby incorporated by reference.

In some embodiments, the dystrophin gene may be provided in a targeting vector. The dystrophin gene may be operably linked to a promoter(s), and/or enhancer(s), and/or other regulatory sequences(s). The promoter may be a constitutive or conditional promoter. The promoter may be a muscle cell-specific promoter.

Dysferlin

In certain embodiments, the muscle membrane protein-encoding gene is a dysferlin gene. In some embodiments, this gene is a cDNA derived from dysferlin transcript variant 1 mRNA of Genbank Accession No. NM_{—}001130987.1, dysferlin transcript variant 2 mRNA of Accession No. NM_{—}001130455.1, dysferlin transcript variant 3 mRNA of Accession No. NM_{—}001130986.1, dysferlin transcript variant 4 mRNA of Accession No. NM_{—}001130985.1, dysferlin transcript variant 5 mRNA of Accession No. NM_{—}001130984.1, dysferlin transcript variant 6 mRNA of Accession No. NM_{—}001130983.1, or dysferlin transcript variant 7 mRNA of Accession No. NM_{—}001130982.1. In certain embodiments, the term “dysferlin gene” refers to a nucleic acid sequence encoding a dysferlin protein of Genbank Accession No. AAC63519.1, Accession No. CAD92859.1, Accession No. ABI75150.1, or Accession No. CAA07800.1.

In some embodiments, the dysferlin gene may be provided in a targeting vector. The dysferlin gene may be operably linked to a promoter(s), and/or enhancer(s), and/or other regulatory sequences(s). The promoter may be a constitutive or conditional promoter. The promoter may be a muscle cell-specific promoter.

Reprogramming Transcription Factors

A reprogramming transcription factor (TF) that may be introduced into a cell isolated from a subject may be any TF that can induce a non-pluripotent cell, such as a somatic cell (e.g. a fibroblast cell) or a mesenchymal stem cell to form an induced pluripotent stem cell. Examples of such TFs include, an Oct family gene, a Sox family gene, a Myc family gene, a Klf family gene, a Nanog family gene, a Lin28 family gene or a NR5A (nuclear receptor subfamily 5, group A) family gene. Examples of TF genes from the Oct family include Oct4, Oct1A, Oct6, and the like. Examples of TF genes from the Sox family include Sox1, Sox-2, Sox3, Sox7, Sox15, Sox17 and Sox18. Examples of the TF genes from the Klf (Kruppel like factor-4) family gene include Klf1, Klf2, Klf4, Klf5 and the like. Examples of TF genes from the Myc family gene include c-Myc, N-Myc, L-Myc and the like. Lin28 family genes include, for example, Lin28 and Lin28b. An example of TF genes from the NR5A family is NR5A2.

A nucleic acid sequence comprising one or more nucleic acid sequences encoding different reprogramming transcription factors may be introduced into a cell isolated from a subject. In certain embodiments, a nucleic acid sequence encoding a single reprogramming transcription factor may be introduced. In other embodiments, a nucleic acid sequence encoding two reprogramming transcription factors may be introduced. In certain cases, first reprogramming TF and second reprogramming TF-encoding sequences may be introduced. The first reprogramming TF may be a TF from the Oct family, e.g., Oct4 and second reprogramming TF may be of the Sox family, e.g., Sox2. In certain cases, a first, second, and a third reprogramming TF-encoding sequence may be introduced into a cell isolated from a subject. The three TFs may be Oct4, Sox2, and c-Myc. In certain cases, a first, second, a third, and a fourth reprogramming TF-encoding sequence may be introduced into a cell isolated from a subject. The four TFs may be Oct4, Sox2, c-Myc, and Klf4. In certain cases, the third or the fourth TF may be a member of the: Myc family, e.g., c-My; Nanog family, e.g., Nanog; Lin family, e.g., Lin28; Klf family, e.g., Klf4; or NR5A family, e.g., NR5A2.

A nucleic acid encoding a reprogramming transcription factor may be operably linked to a constitutive or a conditional promoter.

In some embodiments, a nucleic acid encoding a reprogramming transcription factor may be flanked by a pair of compatible targeting sites arranged in the same orientation, for example, head to head or tail to tail. The pair of compatible targeting sites facilitates excising the nucleic acid encoding a reprogramming transcription factor, if desired.

As noted above, in certain embodiments the nucleic acid encoding one or more reprogramming transcription factors, may comprise one or more compatible targeting sites that are specific for a unidirectional or a bidirectional recombinase. In some embodiments, the nucleic acid encoding one or more reprogramming transcription factors may be flanked by two compatible targeting sites specific for a bidirectional recombinase, wherein the two compatible targeting sites are arranged in the same orientation. After formation of an induced pluripotent stem cell, the nucleic acid encoding one or more reprogramming transcription factors may be excised by exposing the induced pluripotent stem cell to a recombinase, e.g., a unidirectional recombinase or a bidirectional recombinase that specifically recognizes the targeting sites present on the nucleic acid. In certain embodiments, the targeting sites may be loxP sites and the bidirectional recombinase may be Cre recombinase. In certain embodiments, the unidirectional recombinase may be phiBT, R4, or Bxb1 recombinase. The compatible targeting sites on which these unidirectional recombinases act are described above.

Cell Isolated from a Subject

A cell isolated from a subject may be any cell such as a non-pluripotent cell, for example a somatic cell (e.g., a fibroblast cell) or a mutipotent cell, such as mesenchymal stem cell (e.g. an adipose-derived mesenchymal stem cell). In general, the subject methods involve generating an induced pluripotent stem cell from a cell isolated from a subject. However, in certain embodiments, such as where the isolated cell is a stem cell, e.g., a mesenchymal stem cell, for example, an adipose-derived mesenchymal stem cell, the method does not include generating an induced pluripotent stem cell from a cell isolated from a subject.

Examples of suitable somatic cells that may be used in the methods disclosed herein include, but are not limited to: fibroblasts (e.g., skin fibroblasts, dermal fibroblasts), bone marrow-derived mononuclear cells, muscle cells, peripheral blood mononuclear cells, macrophages, hepatocytes, keratinocytes, oral keratinocytes, hair follicle cells, dermal cells, epithelial cells, gastric epithelial cells, lung epithelial cells, synovial cells, kidney cells, skin epithelial cells, pancreatic beta cells, neuronal cell, retinal cell, glial cell, and osteoblasts, for example.

Non-pluripotent cells such as somatic cells used to generate iPSCs can originate from a variety of types of tissue including but not limited to: bone marrow, skin (e.g., dermis, epidermis), muscle (e.g., skeletal muscle, cardiac muscle, smooth muscle), adipose tissue, peripheral blood, central nervous system tissue (e.g., brain, spinal cord). The cells used to generate iPS cells can also be derived from neonatal tissue, including, but not limited to: umbilical cord tissues (e.g., the umbilical cord, cord blood, cord blood vessels), the amnion, the placenta, and various other neonatal tissues (e.g., bone marrow fluid, muscle, adipose tissue, peripheral blood, skin, skeletal muscle etc.).

Cells used to generate iPSCs can be derived from tissue of a non-embryonic subject, a neonatal infant, a child, or an adult. Cells used to generate iPS cells can be derived from neonatal or post-natal tissue collected from a subject within the period from birth, including cesarean birth, to death. For example, the tissue source of cells used to generate iPS cells can be from a subject who is greater than about 10 minutes old, greater than about 1 hour old, greater than about 1 day old, greater than about 1 month old, greater than about 2 months old, greater than about 6 months old, greater than about 1 year old, greater than about 2 years old, greater than about 5 years old, greater than about 10 years old, greater than about 15 years old, greater than about 18 years old, greater than about 25 years old, greater than about 35 years old, greater than about 45 years old, greater than about 55 years old, or greater than about 65 years old.

The somatic cell may be obtained from mammals such as horse, canines, and primates, such as, humans.

The methods described herein may also be used to convert a non-pluripotent stem cell, e.g., a multipotent cell from a subject into an iPS cell. Examples of multipotent cells that can be induced to form iPS cells include placenta-derived mesenchymal stem cells, adipose-derived stem cells, and the like.

iPS cells produced by the methods disclosed herein may be detected based on the presence of one or more properties including but not limited to expression of particular proteins, an ES cell like morphology, pluripotency, growth properties, epigenetic reprogramming. These properties are described below. In certain embodiments, an iPS cell may possess two or more, or three or more, or four or more, or five or more, or six or more, or more, for example, seven of the following properties. iPS cells may produce and express on their cell surface one or more of the following cell surface antigens: Stage-Specific Embryonic Antigens 3 (SSEA-3) and 4 (SSEA-4), Tumor Rejection Antigens (TRA): TRA-1-60, TRA-1-81, TRA-2-49/6E (alkaline phophatase), and Nanog. iPS cells may express one or more of the following genes: Oct-3/4, Sox2, Nanog, GDF3, REX1, FGF4, ESG1, DPPA2, DPPA4, and hTERT. One or more of the foregoing cell surface markers or other genes expressed by iPS cells may be used to detect iPS cells. In certain embodiments, an iPS cell may be a cell that expresses at least two of the above-mentioned cell surface markers. In certain embodiments, an iPS cell may be a cell that expresses at least three of the above-mentioned cell surface markers. In certain embodiments, an iPS cell may be a cell that expresses at least three of the above-mentioned cell surface markers.

Detection of markers may be achieved through any means known in the art, for example immunologically. Histochemical staining, flow cytometry, fluorescence activated cell sorting (FACS), Western Blot, enzyme-linked immunosorbent assay (ELISA), etc. may be used. Flow immunocytochemistry may be used to detect cell-surface markers, immunohistochemistry (for example, of fixed cells or tissue sections) may be used for intracellular or cell-surface markers. Western blot analysis may be conducted on cellular extracts. Enzyme-linked immunosorbent assay may be used for cellular extracts or products secreted into the medium. Antibodies for the identification of stem cell markers may be obtained from commercial sources, for example from Chemicon International, (Temecula, Calif., USA). The immunological detection of these antigens using monoclonal antibodies has been widely used to characterize pluripotent stem cells (Shamblott M J. et. al. (1998) PNAS 95: 13726-13731; Schuldiner M. et. al. (2000). PNAS 97: 11307-11312; Thomson J. A. et. al. (1998). Science 282: 1145-1147; Reubinoff B. E. et. al. (2000). Nature Biotechnology 18: 399-404; Henderson J. K. et. al. (2002). Stem Cells 20: 329-337; Pera M. et. al. (2000). J. Cell Science 113: 5-10.).

Other than gene expression, iPS cells may be detected by assessing cell morphology, pluripotency or multi-lineage differentiation potential or any characteristics known in the art, or any combination thereof.

Successfully generated iPS cells are remarkably similar to naturally-isolated pluripotent stem cells (such as mouse and human embryonic stem cells, mESCs and hESCs, respectively), thus confirming the identity, authenticity, and pluripotency of iPS cells to naturally-isolated pluripotent stem cells. Thus, induced pluripotent stem cells generated from the subject methods disclosed could be selected based on one or more of following embryonic stem cell characteristics, as outlined below:

A. Cellular Biological Properties

Morphology: iPS cells are morphologically similar to ESCs. Each cell may have round shape, large nucleolus and scant cytoplasm. Colonies of iPS cells maybe also similar to that of ESCs. Human iPS cells form sharp-edged, flat, tightly-packed colonies similar to hESCs and mouse iPS cells form the colonies similar to mESCs, less flatter and more aggregated colonies than that of hESCs.

Growth properties: Doubling time and mitotic activity are cornerstones of ESCs, as stem cells must self-renew as part of their definition. iPS cells may be mitotically active, actively self-renewing, proliferating, and dividing at a rate equal to ESCs.

Stem Cell Markers: iPS cells may express cell surface antigenic markers expressed on ESCs. Human iPS cells expressed the markers specific to hESC, including, but not limited to, SSEA-3, SSEA-4, TRA-1-60, TRA-1-81, TRA-2-49/6E, and Nanog. Mouse iPS cells expressed SSEA-1 but not SSEA-3 nor SSEA-4, similarly to mESCs.

Stem Cell Genes: iPS cells may express genes expressed in undifferentiated ESCs, including Oct-3/4, Sox2, Nanog, GDF3, REX1, FGF4, ESG1, DPPA2, DPPA4, and hTERT.

Telomerase Activity: Telomerases are necessary to sustain cell division unrestricted by the Hayflick limit of 50 cell divisions. hESCs express high telomerase activity to sustain self-renewal and proliferation, and iPS cells also demonstrate high telomerase activity and express hTERT (human telomerase reverse transcriptase), a necessary component in the telomerase protein complex.

Pluripotency: iPS cells will be capable of differentiation in a fashion similar to ESCs into fully differentiated tissues.

Neural Differentiation: iPS cells could be differentiated into neurons, expressing P3μ tubulin, tyrosine hydroxylase, AADC, DAT, ChAT, LMX1B, and MAP2. The presence of catecholamine-associated enzymes may indicate that iPS cells, like hESCs, may be differentiable into dopaminergic neurons. Stem cell-associated genes will be downregulated after differentiation.

Cardiac Differentiation: iPS cells could be differentiated into cardiomyocytes that spontaneously began beating. Cardiomyocytes expressed TnTc, MEF2C, MYL2A, MYHC 13, and NKX2.5. Stem cell-associated genes will be down-regulated after differentiation.

Teratoma Formation: iPS cells injected into immunodeficient mice may spontaneously formed teratomas after certain time, such as nine weeks. Teratomas are tumors of multiple lineages containing tissue derived from the three germ layers endoderm, mesoderm and ectoderm; this is unlike other tumors, which typically are of only one cell type. Teratoma formation is a landmark test for pluripotency.

Embryoid Body: hESCs in culture spontaneously form ball-like embryo-like structures termed “embryoid bodies,” which consist of a core of mitotically active and differentiating hESCs and a periphery of fully differentiated cells from all three germ layers. iPS cells may also form embryoid bodies and have peripheral differentiated cells.

Blastocyst Injection: hESCs naturally reside within the inner cell mass (embryoblast) of blastocysts, and in the embryoblast, differentiate into the embryo while the blastocyst's shell (trophoblast) differentiates into extraembryonic tissues. The hollow trophoblast is unable to form a living embryo, and thus it is necessary for the embryonic stem cells within the embryoblast to differentiate and form the embryo. iPS cells injected by micropipette into a trophoblast to generate a blastocyst transferred to recipient females, may result in chimeric living mouse pups: mice with iPS cell derivatives incorporated all across their bodies with 10%-90 and chimerism.

In certain embodiments, an iPS cell may be a cell that exhibits at least two of the above-mentioned cellular biological properties, for example, pluripotency and growth properties. In certain embodiments, an iPS cell may be a cell that exhibits at least three of the above-mentioned cellular biological properties, for example, pluripotency, growth properties, and embryoid body formation.

In certain embodiments, an iPS cell may be a cell that expresses at least one of the above mentioned cell surface markers and at least one of the above-mentioned cellular biological properties. For example, an iPS cell may be a cell that expresses SSEA-3, SSEA-4 and is pluripotent.

B. Epigenetic Reprogramming

Promoter Demethylation: Methylation is the transfer of a methyl group to a DNA base, typically the transfer of a methyl group to a cytosine molecule in a CpG site (adjacent cytosine/guanine sequence). Widespread methylation of a gene interferes with expression by preventing the activity of expression proteins or recruiting enzymes that interfere with expression. Thus, methylation of a gene effectively silences it by preventing transcription. Promoters of endogenous pluripotency-associated genes, including Oct-3/4, Rex1, and Nanog, may be demethylated in iPS cells, showing their promoter activity and the active promotion and expression of pluripotency-associated genes in iPS cells.

Histone Demethylation: Histones are compacting proteins that are structurally localized to DNA sequences that can affect their activity through various chromatin-related modifications. H3 histones associated with Oct-3/4, Sox2, and Nanog may be demethylated to activate the expression of Oct-3/4, Sox2, and Nanog.

In certain embodiments, an iPS cell may be a cell that expresses at least one of the above mentioned cell surface markers, at least one of the above mentioned cellular biological properties, and demethylation of promoter regions of endogenous pluripotency-associated genes or of histones associated with endogenous pluripotency-associated genes.

Culturing of iPS Cells. After somatic cells are introduced with nucleic acid sequence(s) encoding one or more reprogramming transcription factors, these cells may be cultured in a medium sufficient to maintain the pluripotency. Culturing of iPS cells can use various medium and techniques developed to culture pluripotent stem cells, specially, embryonic stem cells, as described in U.S. Pat. App. 20070238170 and U.S. Pat. App. 20030211603.

For example, like human embryonic stem (hES) cells, iPS cells can be maintained in 80% DMEM (Gibco #10829-018 or #11965-092), 20% defined fetal bovine serum (FBS) not heat inactivated, 1% non-essential amino acids, 1 mM L-glutamine, and 0.1 mM .beta.-mercaptoethanol. Alternatively, ES cells can be maintained in serum-free medium, made with 80% Knock-Out DMEM (Gibco #10829-018), 20% serum replacement (Gibco #10828-028), 1% non-essential amino acids, 1 mM L-glutamine, and 0.1 mM beta-mercaptoethanol. Just before use, human bFGF may be added to a final concentration of about 4 ng/mL (WO 99/20741).

iPS cells, like ES cells, have characteristic antigens that can be identified by immunohistochemistry or flow cytometry, using antibodies for SSEA-1, SSEA-3 and SSEA-4 (Developmental Studies Hybridoma Bank, National Institute of Child Health and Human Development, Bethesda Md.), and TRA-1-60 and TRA-1-81 (Andrews et al., 1987). Pluripotency of embryonic stem cells can be confirmed by injecting the cells into the rear leg muscles of 8-12 week old male SCID mice. Teratomas that demonstrate at least one cell type of each of the three germ layers confirm that the presence of iPS cells.

After the introduction of a gene encoding a reprogramming transcription factor, the non-pluripotent cell may be cultured for about 1 day, or about 3 days, or about 10 days, or about, 18 days, or about 24 days, or more to produce iPS cells. The non-pluripotent cell culture may be monitored at certain time points to detect the presence of iPS cells.

Disease Conditions

The iPS cells produced using the methods presented herein and additionally modified to include a muscle membrane-encoding gene may be used for treatment of the subject from whom the non-plurioptent cell was isolated.

Similarly, the genetically modified mesenchymal stem cell that includes a muscle membrane-encoding gene may be used for treatment of the subject from whom the mesenchymal stem cell was isolated.

In general, the subject may be diagnosed as having a muscle dystrophy disease associated with deficiency of one or more muscle membrane proteins, such as, for example, reduction in the expression level of a functional muscle membrane protein. Diseases of interest for treatment with the subject methods and compositions, such as AD-MSC genetically modified to express a muscle membrane protein or iPS cells modified to express a muscle membrane protein, include sever and mild muscular dystrophies. For example, Duchenne muscular dystrophy is an X-linked recessive disorder characterized by progressive proximal muscle weakness with destruction and regeneration of muscle fibers and replacement by connective tissue. Duchenne muscular dystrophy is caused by a mutation at the Xp21 locus, which results in the absence of dystrophin. It affects 1 in 3000 live male births. Symptoms typically start in boys aged 3 to 7 yr. Progression is steady, and limb flexion contractures and scoliosis develop. Finn pseudohypertrophy (fatty and fibrous replacement of certain enlarged muscle groups, notably the calves) develops. Most patients are confined to a wheelchair by age 10 or 12 and die of respiratory complications by age 20.

Becker muscular dystrophy is a less severe variant, also due to a mutation at the Xp21 locus. Dystrophin is reduced in quantity or in molecular weight. Patients usually remain ambulatory, and most survive into their 30 s and 40 s.

Limb girdle muscular dystrophy (LGMD) refers to a group of related disorders that are amenable to treatment using the subject methods and compositions. For example, LGMD 2B is caused by deficiency of a muscle membrane protein called dysferlin.

Subjects receiving iPS cells comprising a muscle membrane-encoding gene or AD-MSCs comprising a muscle membrane-encoding gene may be tested in order to assay the efficacy of the subject methods. Significant improvements in one or more of parameters are indicative of efficacy. It is well within the skill of the ordinary healthcare worker (e.g., clinician) to adjust dosage regimen and dose amounts to provide for optimal benefit to the patient according to a variety of factors (e.g., patient-dependent factors such as the severity of the disease and the like, the cell type administered, and the like).

In some embodiments, the subject method results in an increase in muscle fibers, for example, at least about a 2.5-fold increase or more, at least about a 3-fold increase or more, at least about a 3.5-fold increase or more, at least about a 4-fold increase or more, at least about a 4.5-fold increase or more, at least about a 5-fold increase or more, at least about a 5.5-fold increase or more, at least about a 6-fold increase or more, at least about a 6.5-fold increase or more, at least about a 7-fold increase or more, at least about a 7.5-fold increase or more at least about a 8-fold increase or more, and up to about 10-fold increase or more, including about 15-fold increase or more, about 20-fold increase or more, such as 25-fold increase or more of muscle fibers, as compared to a control subject who did not receive the cells. An increase in muscle fibers can be measured by any of a variety of methods well known in the art, for example, muscle strength, diameter, and the like.

Introducing comprises delivering a polynucleotide, such as an expression cassette, a targeting vector, etc., to a cell by any method that is known to persons skilled in the art. These methods include, but are not limited to, any manner of transfection, such as for example transfection employing DEAE-Dextran, calcium phosphate precipitation, cationic lipids/liposomes, micelles, manipulation of pressure, microinjection, electroporation, immunoporation, nucleofection, lipofection, use of vectors such as viruses (e.g., RNA virus), plasmids, cell fusions, and coupling of the polynucleotides to specific conjugates or ligands such as antibodies, antigens, or receptors, passive introduction, adding moieties to the polynucleotide that facilitate its uptake, and the like. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

Engrafting includes administering the cells produced by the methods described herein into the subject from whom the cells were obtained. Engrafting can be carried out by injecting the cells into the subject, for example, intravenously, intra-muscularly, intra-arterially, and the like.

In certain embodiments, engrafting may comprise engrafting about 10², 10⁴, 10⁶, 10⁷, 10⁸, 10⁹, 10¹⁰, 10¹², or more cells. The number of cells engrafted may be chosen based on the route of administration and/or the severity of the condition for which the cells are being engrafted.

In certain embodiments, the AD-MSC or the iPS cell carrying the muscle membrane protein-encoding gene may be differentiated into muscle precursor cells before the engrafting. For example, the genetically modified cells may be differentiated into muscle precursor cells such as satellite cells and myoblasts. Differentiation may be carried out using a number of protocols. For example, overexpressing insulin-like growth factor-2 in the stem cells and culturing the cells in the presence of dimethyl sulfoxide to generate skeletal muscle cells, see, e.g., Prelle et al., (2000), Biochem. Biophys. Res. Commun., 277:631-63; using selective culture conditions and FACS sorting to purify skeletal myoblasts (Barberi et al 2007, Nature Medicine 13:642-648); expressing Pax3 in the genetically modified cells, sorting for the PDGF-α a receptor, a marker of early mesoderm, and for the absence of FLK-1, a marker of late mesoderm, a population of cells were derived that gave rise to muscle fibers in vitro and in vivo (Darabi, R. et al., Nature Medicine 14:134-143, 2008); enriching for Pax7-positive satellite-like cells; FACS sorting with a novel anti-satellite cell antibody, SM/C-2.6 (Chang, H., et al. 2009. FASEB Journal 23:1907-1919); overexpressing MyoD in the genetically modified cells, culturing in standard culture conditions for promoting muscle cell growth.

Standard markers for myoblasts such as MyoD and Pax7 may be used to confirm the generation of myoblasts. Formation of satellite cells may be detected by the presence of markers, such as, PAX7 and Pax3. Activated satellite cells may be detected by expression of myogenic transcription factors, such as MyfS and MyoD.

### Compositions

Also provided are compositions comprising autologous cells isolated from a subject, wherein the cells comprise a muscle membrane protein-encoding gene integrated into a genome attachment site in the genome of the cell. The autologous cell may be an induced pluripotent cell or a mesenchymal stem cell, such as, an AD-MSC.

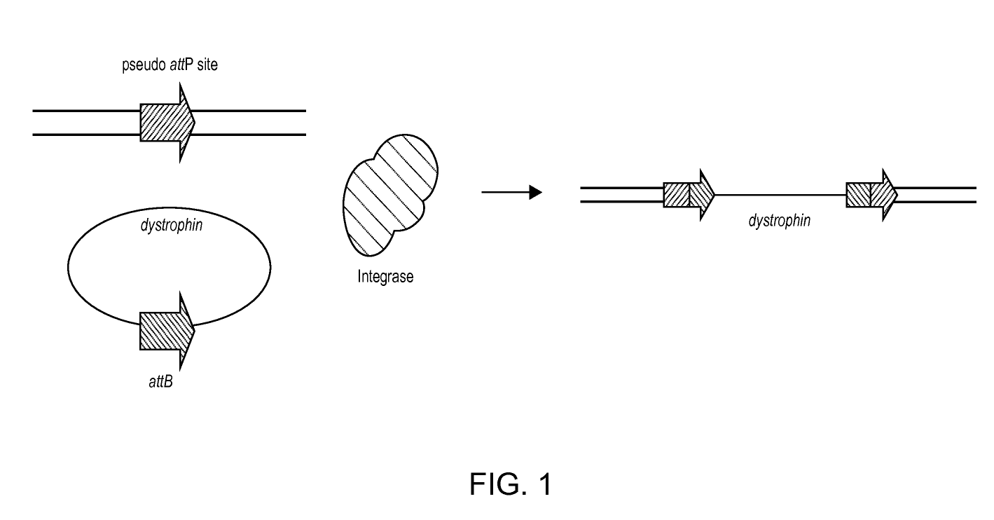

In certain embodiments, the genome attachment site or the genome target site may be an endogenous genome attachment site or one that has been engineered into the genome of the cell by the methods described above. In certain embodiments, the genome attachment site or the genome target site may be an endogenous genome attachment site, for example, the genome attachment site may be one specific for φC31 integrase. For example, the genome attachment site may be pseudo attP site, as described above. In certain embodiments, the composition may comprise an AD-MSC isolated from a subject, wherein the AD-MSC comprises a muscle membrane protein-encoding gene integrated into the genome of the cell at a pseudo attP site, i.e., the gene is flanked by an attR and an attL site. An example of such a cell is provided in FIG. 1. In some embodiments, the composition may comprise a fibroblast cell that has been induced to form iPSC, wherein the cell comprises a muscle membrane protein-encoding gene integrated into the genome of the cell at a pseudo attP site. In some embodiments, the iPSC has been differentiated into a muscle cell or a muscle precursor cell.

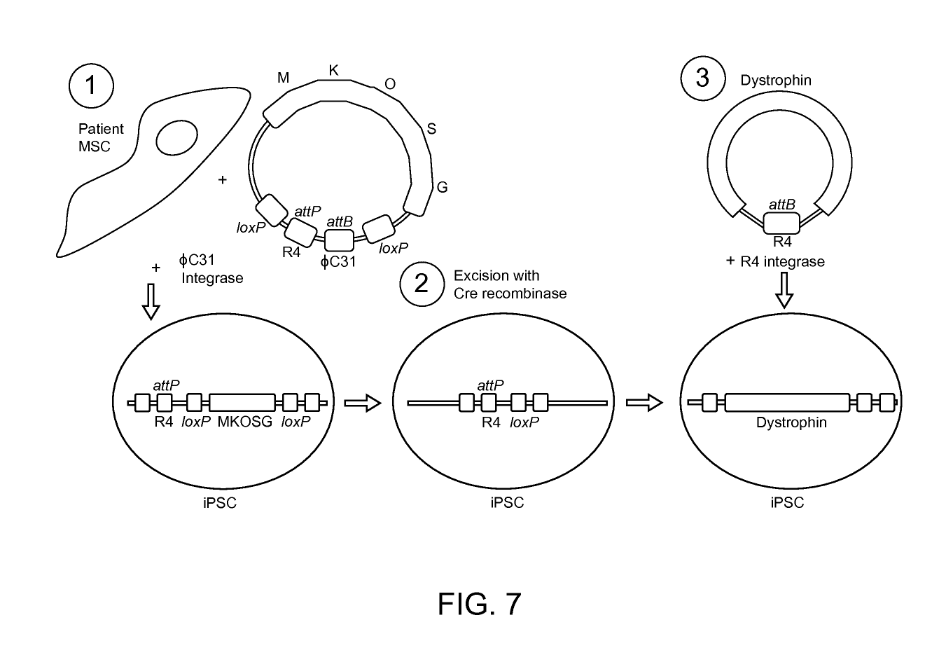

In certain embodiments, the genome attachment site or the genome target site may be an endogenous genome attachment site or one that has been engineered into the genome of the cell by the methods described above. For example, the genome attachment site may be an attP site specific for R4 integrase or a target site specific for Bxb1 recombinase that has been integrated into an endogenous genome attachment site, for example, an attP site, in the genome of the autologous cell. In certain embodiments, the muscle membrane protein-encoding gene may be adjacent to a target site for a bidirectional recombinase, for example a lox site, such as a loxP site. In certain embodiments, the composition may comprise an induced pluripotent stem cell generated from a cell isolated from a subject, wherein the induced pluripotent stem cell comprises a muscle membrane protein-encoding gene integrated into the genome of the cell at a genome attachment site for R4 integrase or Bxb1 recombinase, which genome attachment site is integrated into a pseudo attP site. An example of such a cell is provided in FIG. 7.

The composition may be present in a liquid form or frozen form. The composition may further comprise a pharmaceutically acceptable excipient, such as a buffer, culture medium, stabilizing agent, anti-freeze agent, or the like.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

The following materials and methods were used in the Examples below.

### Cell Culture

Mouse embryonic fibroblasts (MEFs) were prepared from embryonic day 13.5 (E13.5) embryos (C57Bl/6) and cultivated in Dulbecco's modified Eagle's medium high glucose supplemented with 10% fetal bovine serum (FBS), 100 U/ml penicillin, and 100 lg/ml streptomycin (Gibco, Carlsbad, Calif.). ASCs were isolated from the inguinal fat pads of 8-10 weeks old mice (C57Bl/6). Briefly, dissected fat pads were minced and subsequently digested in 0.1% collagenase type IV (Worthington, Lakewood, N.J.) at 37° C. for 1 hour. After separation of adipocytes by centrifugation at 400 g for 10 minutes and filtration through a 100-lm filter mesh, cells were plated onto 10 cm dishes in the same medium used for MEFs. After 24 hours, cells were moved into incubators providing physiological oxygen conditions (5% O2; Sanyo, Wood Dale, Ill.). Medium was changed daily until the first passage of the cells. By using flow cytometry, ASCs were confirmed to be >90% CD29⁺ and Sca-1⁺ and >95% CD34⁻ (Stem cells manuscripts FIG. S1A). To validate the isolation of bona fide ASCs, differentiation ability along mesodermal lineages was assessed. ASCs were differentiated into the osteogenic and adipogenic lineages as shown by alizarin red and oil red 0 staining, respectively (Stem cells manuscripts FIG. S1B). All iPSC lines were maintained on a mitomycin C-treated MEF feeder layer plated on 0.1% gelatin in ESC medium containing 20% ESC-qualified FBS (Invitrogen, Carlsbad, Calif.), 1× nonessential amino acids, 55 μM 2-mercaptoethanol, 100 U/ml penicillin, 100 μg/ml streptomycin, and 10 ng/ml leukemia inhibitory factor (Millipore, Billerica, Mass.). Murine ESCs from strains C57BL/6 and 129 constitutively expressing emerald green fluorescent protein from Invitrogen were used as positive controls.

### Plasmid Construction

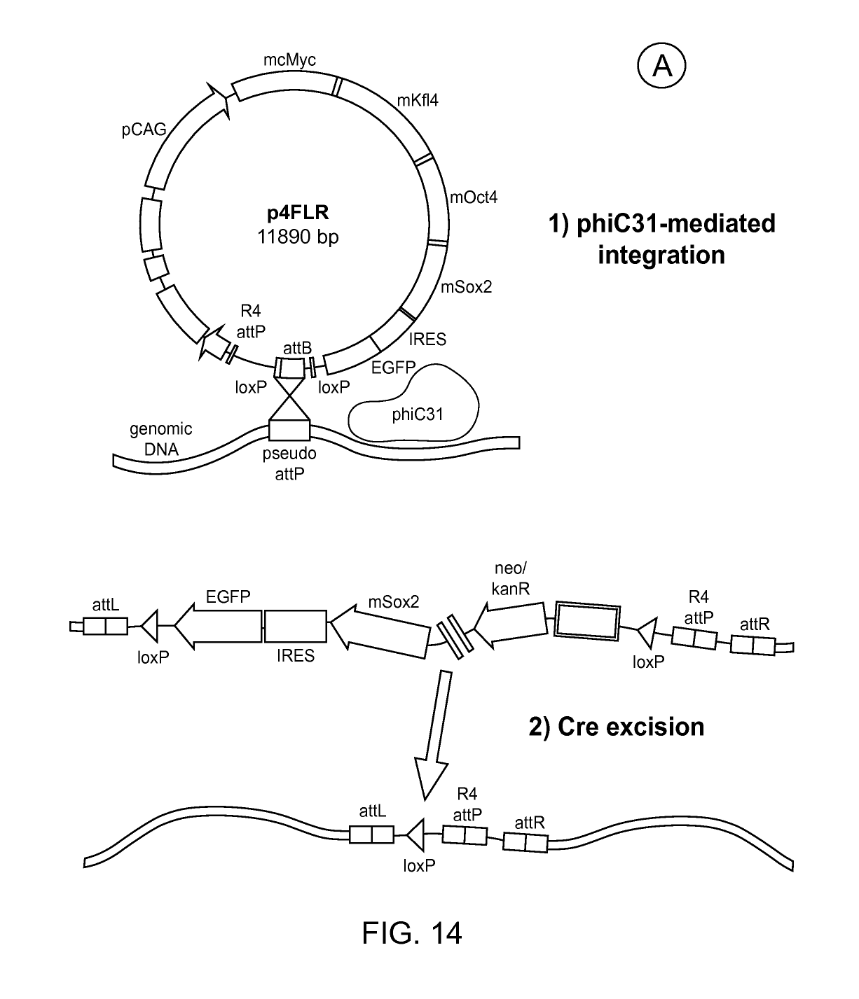

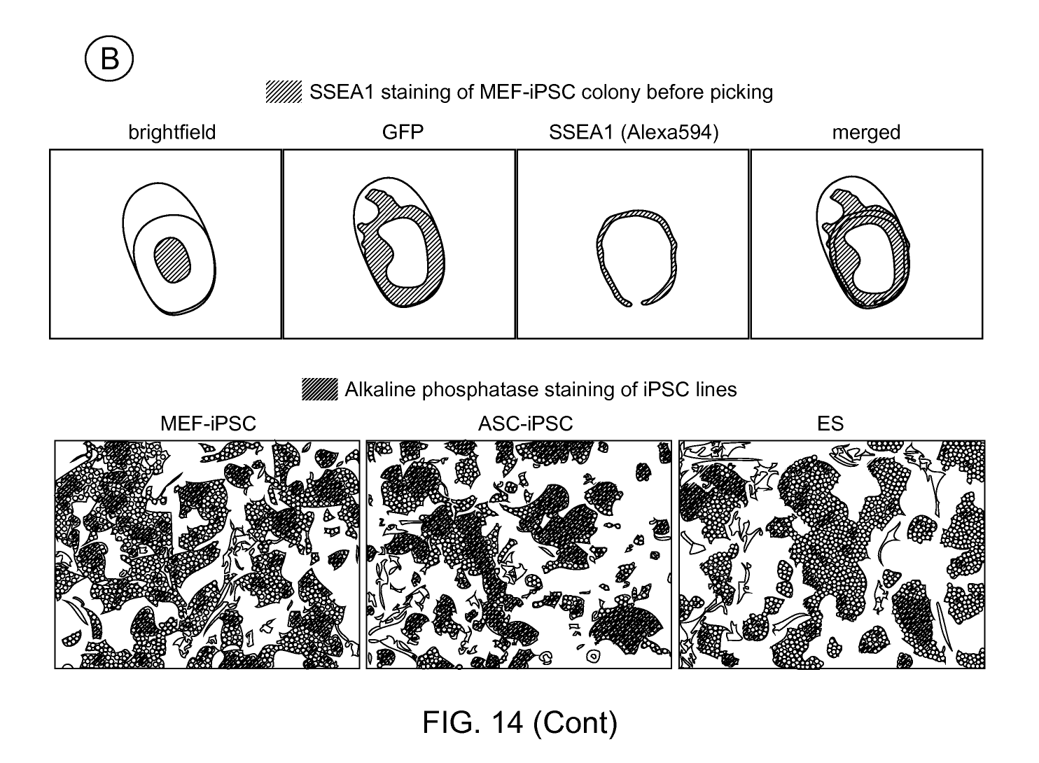

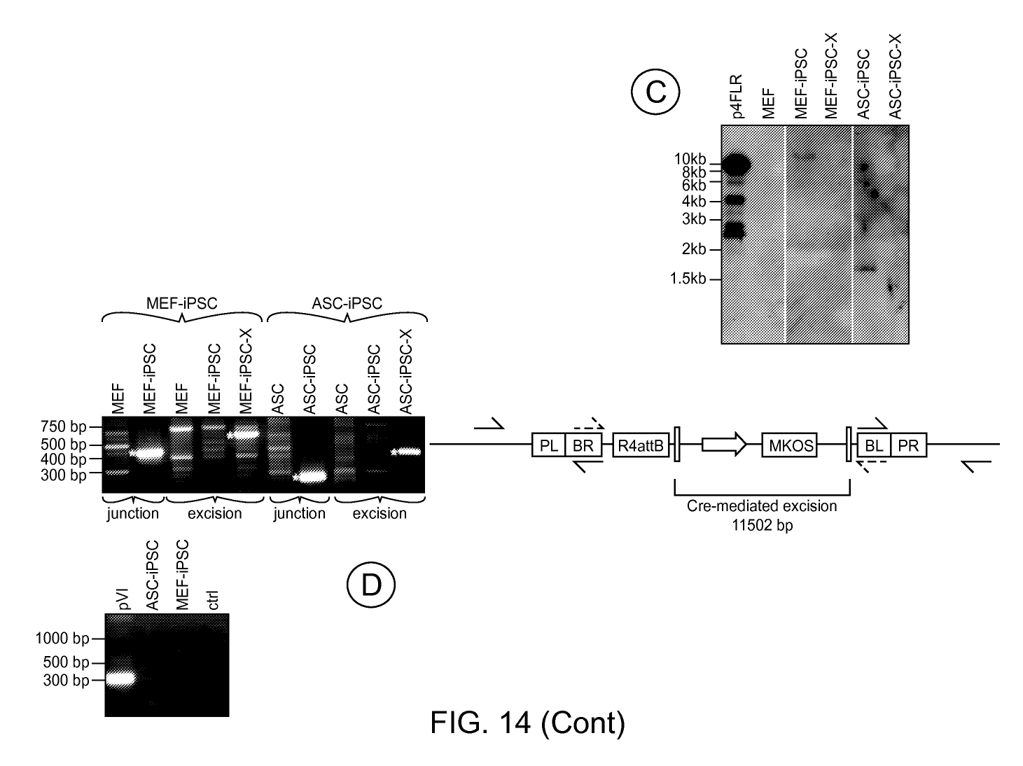

The reprogramming plasmid p4FLR (FIG. 14, Panel A), containing the four Yamanaka factor genes cMyc, Klf4, Oct4, and Sox2 and the EGFP gene, all expressed from a CMV early enhancer/chicken beta actin (CAG) promoter, and a series of recombinase recognition sites, was cloned by using adaptor ligation and a series of polymerase chain reactions (PCRs). A 415-bp fragment carrying the φC31 attB site and R4 attP site, flanked by loxP sites, was synthesized and used in the construction. The reprogramming genes were derived from plasmid PB-TETMKOS. The enhanced green fluorescent protein (EGFP) sequence and plasmid backbone were derived from pEGFP-1 (Clontech, Palo Alto, Calif.), which carries a neomycin/kanamycin resistance gene under the control of the SV40 early promoter.

The sequence of p4FLR includes 11,884 bp. Both plasmids pVI, expressing wild-type φC31 integrase, and pVmI, expressing nonfunctional φC31 integrase, have been described elsewhere. Plasmid pCAG-Cre, expressing the Cre recombinase gene, was purchased from Addgene (www.addgene.org).

### Nucleofection and Reprogramming

A total of 1×10⁶ each of MEFs or ASCs were nucleofected (Lonza, Walkersville, Md.) according to the manufacturer's instructions using MEF nucleofector kit I (program T-20) or human MSC nucleofector kit (program U-23), respectively. One nucleofection was sufficient; multiple nucleofections were not required. Upon nucleofection with 3 μg total DNA (pVI:p4FLR ratio 1:1 by mass), ASCs were cultivated under low oxygen conditions (5% O₂) for 48 hours. On day 2, 1-3×10⁵ MEFs or ASCs were transferred from uncoated plastic six-well plates onto a mitomycin C-treated MEF feeder layer plated on 0.1% gelatin on 10 cm dishes. Medium was changed every other day. For MEF reprogramming, cells were maintained in an atmospheric oxygen incubator for 10 days after nucleofection, then transferred to a low oxygen incubator (5% O₂) for 2 weeks. Colonies were visible starting from days 8 to 12 and picked between days 20 and 26.

Introduction of Cre in iPSC

Lipofection of iPSCs with pCAG-Cre was performed by using Effectene (Qiagen, Valencia, Calif.). For this purpose, 1 μg DNA was diluted in 100 μl EC buffer and mixed with 3.2 μl enhancer solution provided in the kit. Upon 10 minutes incubation at room temperature, 8 μl effectene reagent was added, and incubated for a further 15 minutes. This transfection mix was added to 2×10⁵ cells plated on 0.1% gelatin. Medium was changed after 48 hours.

### Immunofluorescence and Cell Staining

Cells grown on four-well glass chamber slides (Millipore) were fixed with 4% paraformaldehyde and immunostained with anti-Oct4 (all Abcam, Cambridge, Mass., 1:200 dilution), anti-SSEA-1 (Scbt, Santa Cruz, Calif., 1:100 dilution), anti-Nanog, anti-Sox2, or anti-GFP (Rockland, Gilbertsville, Pa.) and the respective secondary antibodies labeled with Alexa594 or Alexa488 (Invitrogen, Carlsbad, Calif.) in buffer (PBS, 3% BSA, 1% Triton X-100). For counterstaining of the nuclei, 4′,6-diamidino-2-phenylindole, dihydrochloride (DAPI) was included in the mounting medium (ProLong Gold; Molecular Probes, Carlsbad, Calif.). Alkaline phosphatase staining was performed according to the manufacturer's instructions (Stemgent, Cambridge, Mass.). Images of stained sections were taken on an Axioshop 2 Plus microscope with an AxioCam MRc camera (Zeiss, Thornwood, N.Y.).

### In Vitro Differentiation

For in vitro differentiation of iPSCs, embryoid bodies were formed within 3-6 days by transfer into suspension culture on nontissue culture-treated 10 cm plates. To allow spontaneous differentiation, cells were grown in ESC medium in the absence of leukemia inhibitory factor (LIF). After transfer from suspension culture onto 0.1% gelatin-coated 60 mm dishes, days 10-14 embryoid bodies were stained for the respective markers of the three germ layers. Anti-smooth muscle actin (SMA; Sigma, St. Louis, Mo.), anti-a-fetoprotein (AFP, Scbt, Santa Cruz, Calif.), and anti-beta III tubulin (Tuj1, Scbt, Santa Cruz, Calif.) were used. Nuclei were counterstained with Hoechst 33342 (Invitrogen, Carlsbad, Calif.).

### Teratoma and Chimera Formation

Teratoma formation and chimera formation were carried out at the Transgenic Service Center of the Comprehensive Cancer Center at Stanford University School of Medicine. To generate teratomas, 1-2×10⁶ iPSCs generated from a C57BL/6 background were mixed 1:1 with Matrigel (BD Biosciences, San Diego, Calif.) and injected into the kidney capsules of 8 week-old immunodeficient severe combined immunodeficiency (SCID) beige mice. After 4 weeks, tumors were subjected to histological analyses. To form chimeric mice, iPSCs were injected into the blastocysts of albino B6 mice and implanted into the uteri of pseudopregnant foster mothers using routine techniques Chimerism was revealed by the development of black coat color on the host white coat color background. Mice were housed and maintained in the Research Animal Facility at Stanford University in accordance with the guidelines of the Administrative Panel on Laboratory Animal Care of Stanford University.

### Quantitative RT-PCR Analyses

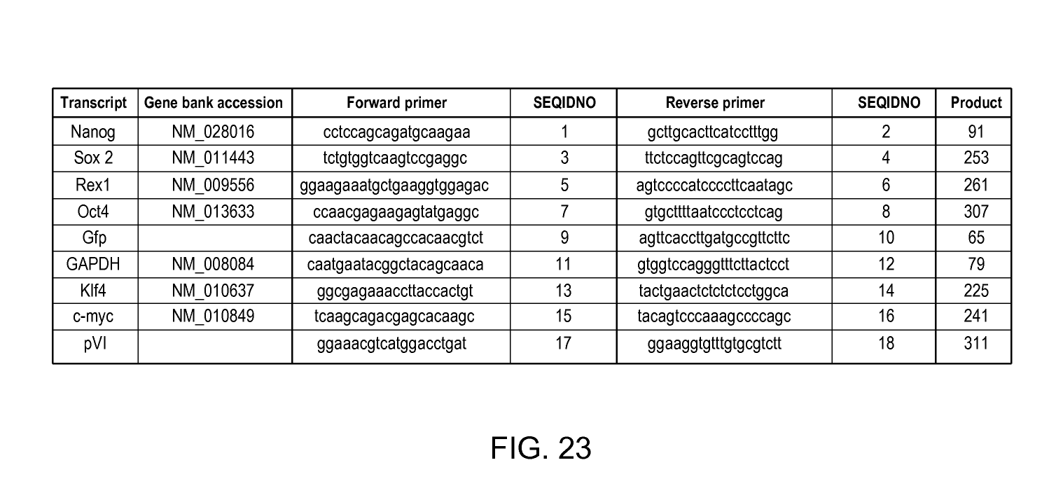

Total RNA was prepared using the RNeasy Mini Plus kit (Qiagen, Valencia, Calif.) and subsequently 1 μg of the RNA was used for reverse transcription using the iScript cDNA synthesis kit (BioRad, Hercules, Calif.), following the manufacturer's instructions. mRNA expression levels were analyzed using iQ SYBR green supermix (BioRad, Hercules, Calif.) and the real time-PCR (RT-PCR) detection system CFX96 (BioRad). Expression levels of individual transcripts (Klf4, cMyc, GFP, Oct4, Sox2, Rex1, and Nanog) were normalized to glyceraldehyde 3-phosphate dehydrogenase (GAPDH) expression and compared with the expression levels in mouse ESCs (mESCs). Primers and PCR conditions are listed further below in the Materials and Methods Section (see also FIG. 23).

### Bisulfite Mutagenesis and Analysis

Primers developed by EpigenDx (Worcester, Mass.) were used to analyze CpG sites within the proximal promoter regions of the murine Oct4 and Nanog promoters. Genomic DNA (1 μg), which was extracted using the DNeasy Blood and Tissue kit (Qiagen, Valencia, Calif.), was sent to Epigendx for bisulfite treatment, PCR, and pyrosequencing.

### Southern Blotting

Genomic DNA (10 μg) from iPSC lines were digested overnight with HindIII and resolved by agarose gel electrophoresis. After transfer and UV crosslinking onto Hybond N+nylon membrane (GE Healthcare, Piscataway, N.J.), the DNA was hybridized with an EGFP probe generated by DIG High Prime Labeling and Detection Starter Kit II (Roche, Indianapolis, Ind.).

### Linker-Mediated Polymerase Chain Reaction

Genomic DNA of 1 μg was digested with MseI overnight (10 μl total reaction), followed by heat inactivation of the enzyme at 65° C. for 20 minutes. The linker (antisense 5′-/5Phos/TAG TCC CTT AAG CGG AG/3AmMO/-3′ SEQ ID NO: 19); sense 5′-GTA ATA CGA CTC ACT ATA GGG CTC CGC TTA AGG GAC-3′ (SEQ ID NO:20); Integrated DNA Technologies, San Diego, Calif.) was ligated with T4 ligase to the entire digest at a final concentration of 0.7 μM at 16° C. overnight. The first round of the nested PCR used linker primer-1 (5′-GTA ATA CGA CTC ACT ATA GG*G*C-3′ (SEQ ID NO:21)) and either attB-F2 (5′-ATG TAG GTC ACG GTC TCG AA*G*C-3′ (SEQ ID NO:22)) or attB-R1 (5′-TCC CGT GCT CAC CGT GAC C*A*C-3′ SEQ ID NO:23)). The second round of the nested PCR used 2 μl of the product from the first round plus linker primer-2 (5′-AGG GCT CCG CTT AAG GG*A*C-3′ (SEQ ID NO:24)) and either attB-F3 (5′-cga agc cgc ggt g*c*g-3′ (SEQ ID NO:25)) or attB-R2 (5′-ACT ACC GCC ACC TCG*A*C-3′ (SEQ ID NO:26)) to amplify the integration junctions. The asterisk is used to denote a phosphorothioate bond. PCR conditions used were 98° C. for 2 minutes, 10 cycles of 98° C. for 15 seconds, 60° C.-55° C. for 30 seconds with 0.5° C. per cycle decrements, 72° C. for 30 seconds, and 30 cycles of 98° C. for 15 seconds, 55° C. for 30 seconds with 72° C. for 30 seconds, and a final elongation at 72° C. for 2 minutes. Upon column purification (Zymoclean Gel Recovery Kit, Zymo Research, Irvine, Calif.) fragments were cloned into the blunt-end vector pJET (Fermentas, Glen Burnie, Md.) according to the manufacturer's instructions. DNA sequencing was performed by Elimbiopharm (Hayward, Calif.) using standard techniques.

Primers and PCR Conditions Used for qRT-PCR and the Detection of pVI

FIG. 23 is a table showing a list of the primers used for qRT-PCR. The primers for Klf4, cMyc, Oct4, Sox2, Rex1, and Nanog were described in Woltjen et al., Nature 2009; 458:766-770. The primers for Oct4, Sox2, Klf4, and cMyc are directed only against the endogenous transcripts, not the plasmid-encoded ones. PCR conditions were 95° C. for 15 min, 40 cycles of 94° C. for 10 sec, 57° C. for 20 sec, 72° C. for 30 sec. The program ended with a melting curve from 65° C. to 95° C. in 0.5° C./cycle increments. PCR conditions for the pVI PCR were 94° C. for 1 min, 30 cycles of 94° C. for 30 sec, 55° C. for 30 sec, 72° C. for 10 sec, and final 72° C. for 1 min

### Differentiation of ASC

To differentiate ASC isolated from the inguinal fat pads of adult mice into the mesodermal adipogenic and osteogenic lineages, cells were grown in differentiation media. Osteogenic differentiation was induced by adding 1 nM dexamethasone, 50 μM ascorbic acid, and 20 mM beta-glycerol phosphate to the culture medium for two weeks. To induce adipogenic differentiation, 1 μM dexamethasone, 500 nM IBMX, 50 μM indomethacine, and 5 μg/ml insulin were added to the culture medium. It was used as induction medium for five days, followed by a two-day period with maintenance medium containing 5 μg/ml insulin for a total of two cycles. Calcification was assessed by Alizarin Red staining after two weeks of differentiation. Cells were washed with PBS three times, followed by a fixation period in 70% Ethanol at −20° C. Upon rehydration with water, cells were stained with a 40 mM Alizarin Red S (Spectrum, Gardena, Calif.) solution (pH 4.1) for 10 minutes. Lipid vacuoles obtained after two weeks in adipogenic differentiation media, were assessed via Oil Red 0 staining. Fixation of the cells was performed by a 30-minute incubation period in formalin fixative. Upon rehydration with water cells were stained with Oil Red 0 (Sigma) staining solution (dilute 0.3% Oil Red 0 in isopropanol stock solution 3:2 in water) for 30 minutes at 37° C. and subsequently washed with PBS two times.

### Flow Cytometry

To analyze the surface marker expression on ASC, antibodies against CD29 (BioLegend, San Diego, Calif.), Sca-1 (BioLegend), and CD34 (Scbt, Santa Cruz, Calif.) were used. Cells were trypsinized and resuspended in FACS buffer (HBSS, 2% FBS) for staining for 20 minutes at 4° C. All antibodies including the isotype control (BioLegend) were directly conjugated to FITC. To determine the nucleofection efficiency, cells were trypsinized 48-72 hours after nucleofection and analyzed using a Scanford flow cytometer (Custom Stanford and Cytek upgraded FACScan) which was also used to detect the expression of the proteins. The flow cytometer was used in the shared FACS facility at Stanford University.

### Chromosome Counts

Metaphase spreads were prepared according to standard protocols (http://web.mitedu/ki/facilities/transgenic/methods/karyotyping.html). Analysis was performed using ImageJ software, counting an average of 50 spreads per clone.

LM-PCR Results for iPSC Clones

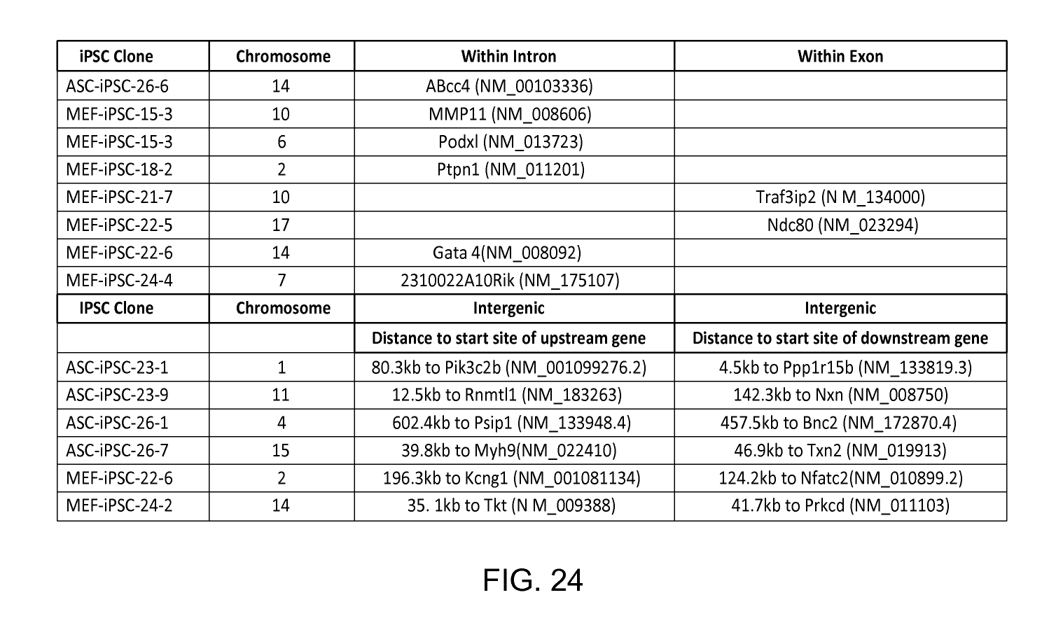

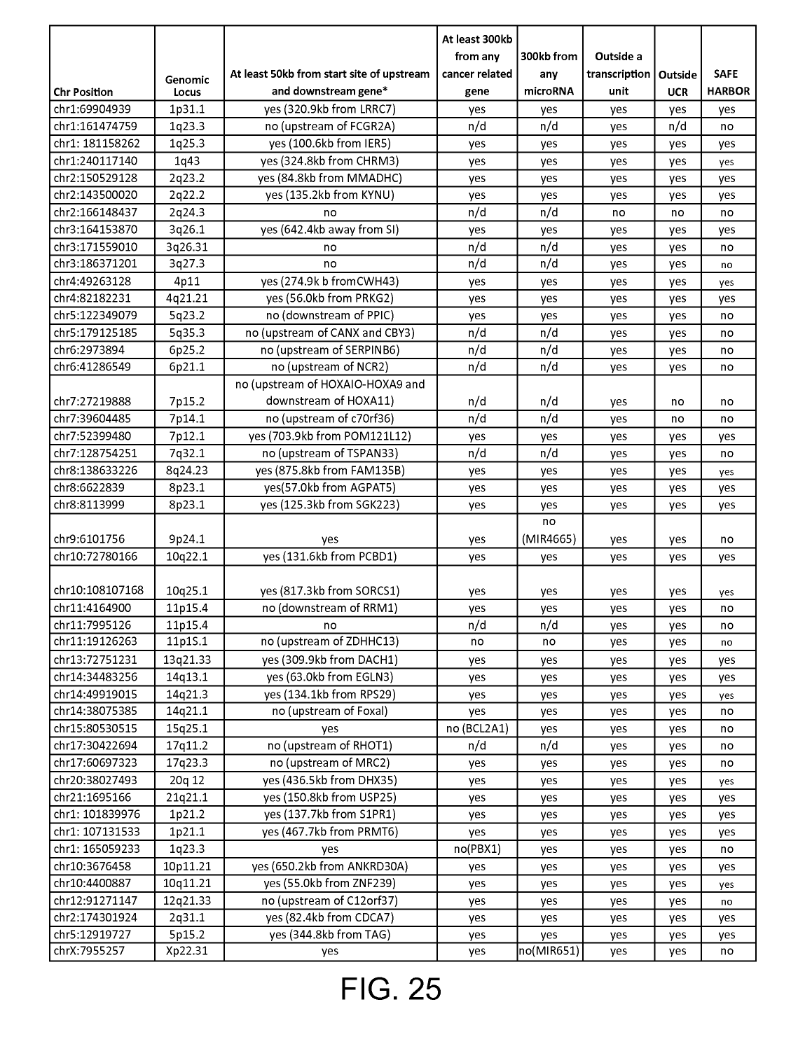

FIG. 24 shows a table that provides an overview of the 14 integration events analyzed by LM-PCR and sequencing. LM-PCR was performed on 14 different iPSC lines and the results are shown in FIG. 24. The chromosome associated with each site is indicated. In the upper half, the intronic and exonic integration sites are further described with the gene name and the gene bank accession number. The lower half shows the six intergenic sites and indicates the distance of each integration site to the promoters of the respective upstream and downstream genes. The two iPSC lines shaded in gray are located more than 50 kb from the start site of any gene, upstream or downstream of the integration site. Moreover, these two integration sites meet all the criteria outlined in Papapetrou et al., Nat. Biotechnol. 2011; 29:73-78, as described in the text, and represent genomic safe harbors.

### Verification of ASC Origin

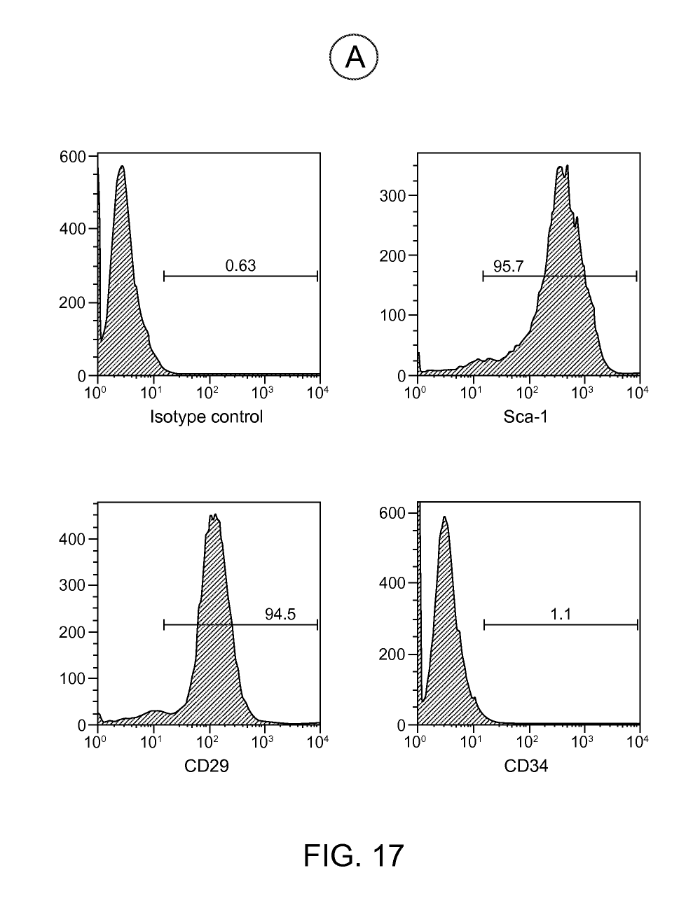

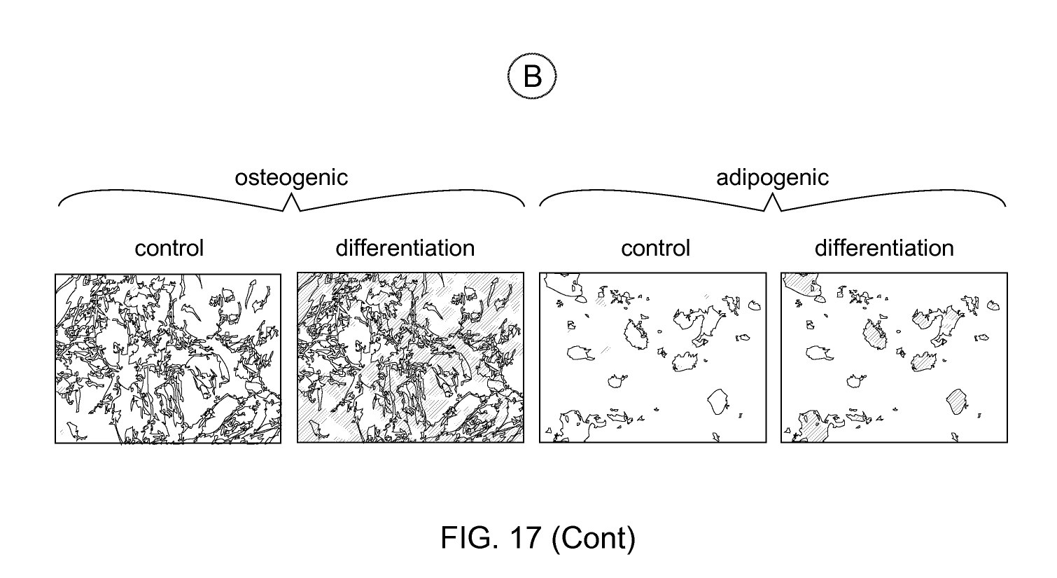

By using flow cytometry, ASC were confirmed to be >90% CD29+ and Sca-1+ and >95% CD34− (FIG. 17, Panel A). Furthermore, the capacity to differentiate along the mesodermal lineage into osteogenic and adipogenic cells was verified. Calcification (FIG. 17, Panel B, left panel) during the osteogenic and formation of lipid vacuoles (FIG. 17, Panel B, right panel) during the adipogenic differentiation, were visualized by Alizarin Red and Oil Red 0 staining, respectively.

Nucleofection Efficiency of p4FLR

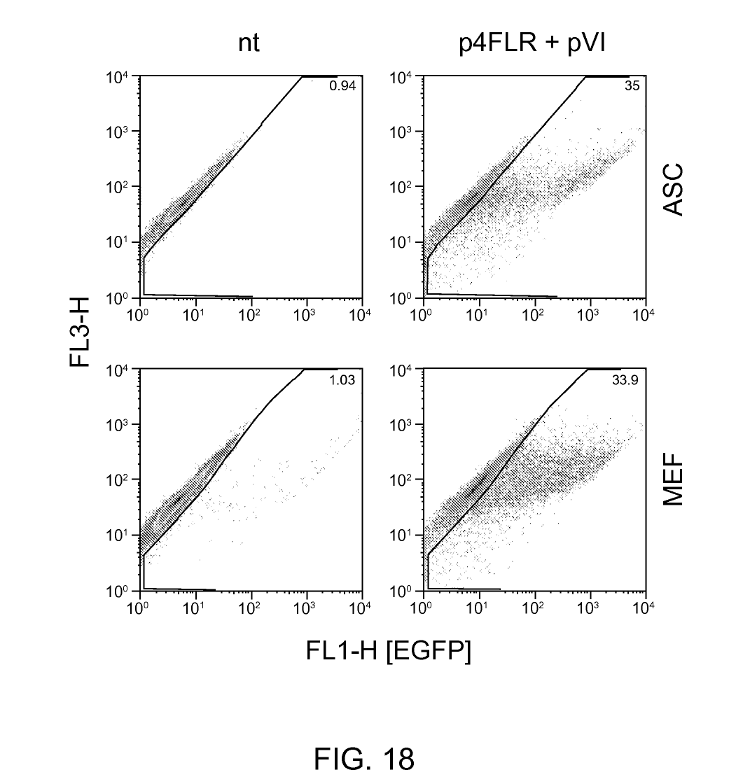

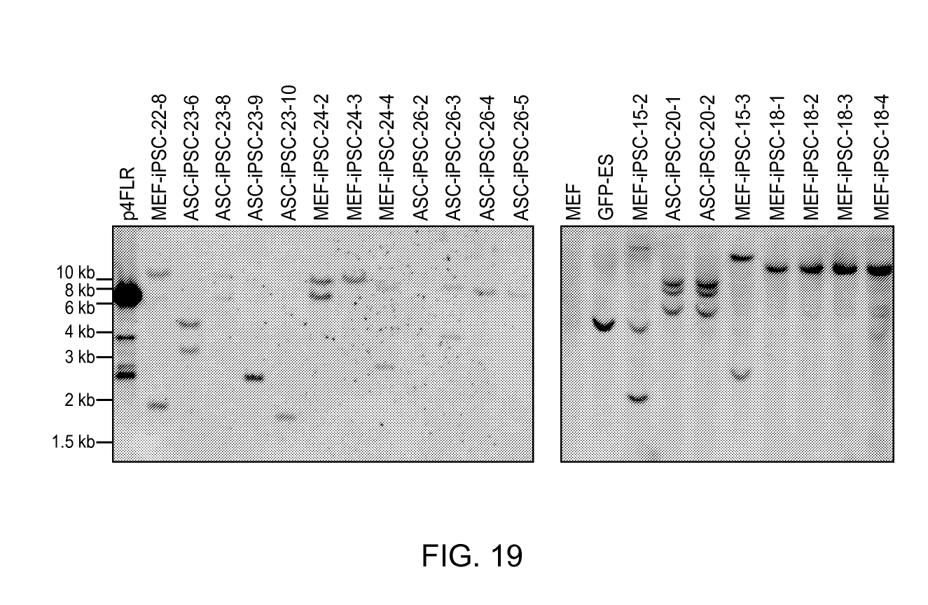

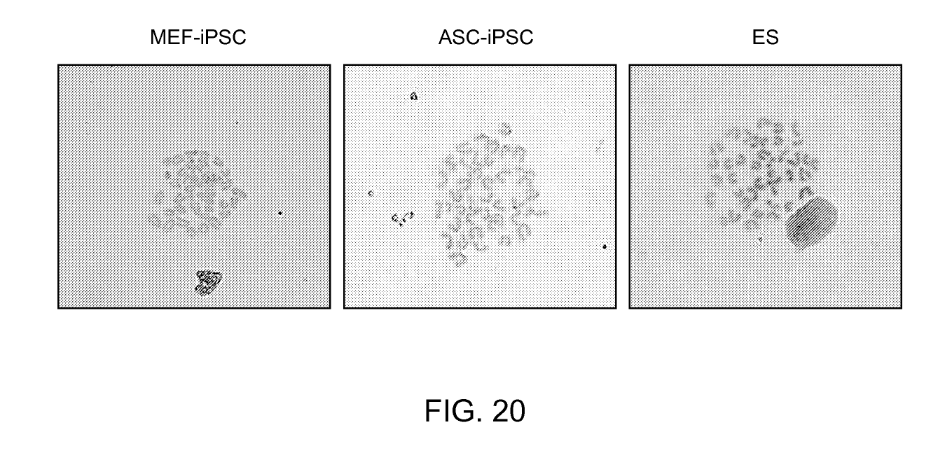

Flow cytometry was performed 48-72 hours after nucleofection of MEF and ASC. Nucleofection efficiencies were quantified via the percentage of GFP positive cells and ranged between 35-64% (FIG. 18). Analysis of integration events. By using Southern blots, single, double, and triple integrants were identified (FIG. 19) Chromosome counts of iPSC. Metaphase spreads of MEF- and ASC-lines were counted and compared to ES cells (FIG. 20).

### Analysis of Oct4 and Nanog Promoter Methylation Status

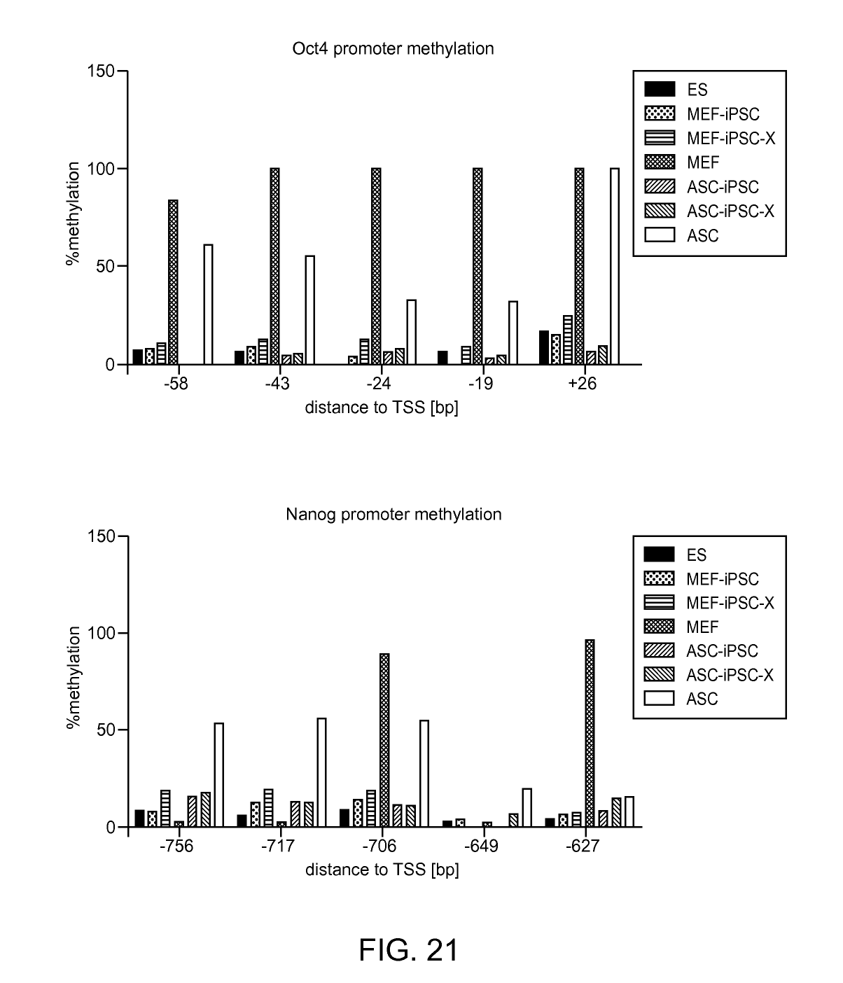

A quantitative evaluation of the methylation of CpG sites within the promoter regions of Oct4 and Nanog was performed using bisulfite pyrosequencing. The methylation status of iPSC and iPSC-X was revealed to be comparable to pluripotent ES cells and different from the starting populations of MEF and ASC (FIG. 21).

Pluripotency of iPSC Before and after Cre-Mediated Excision

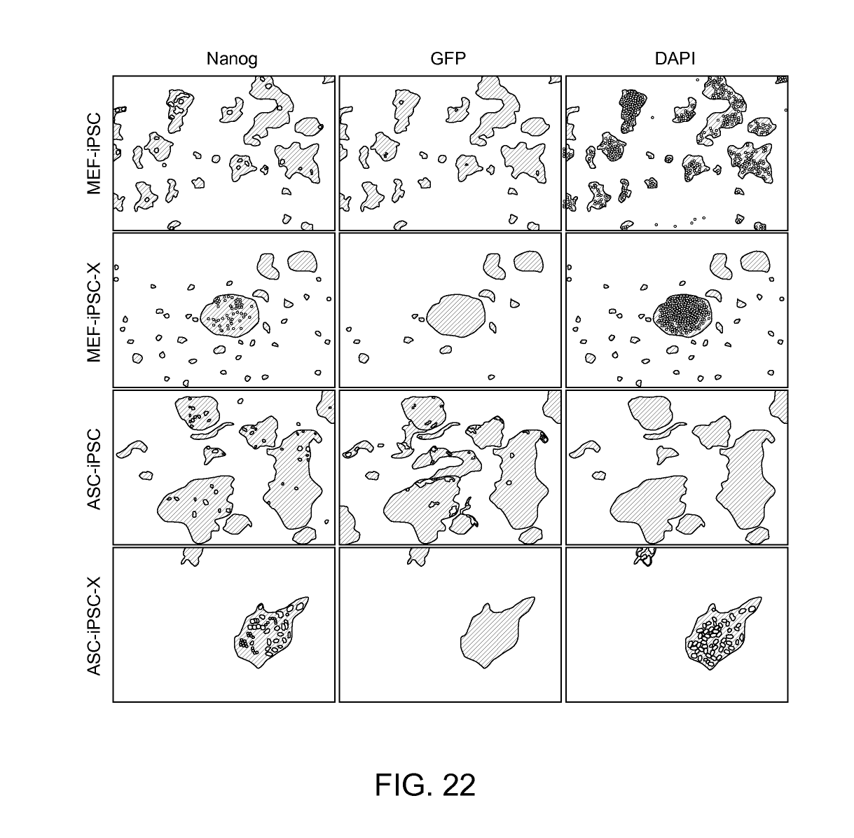

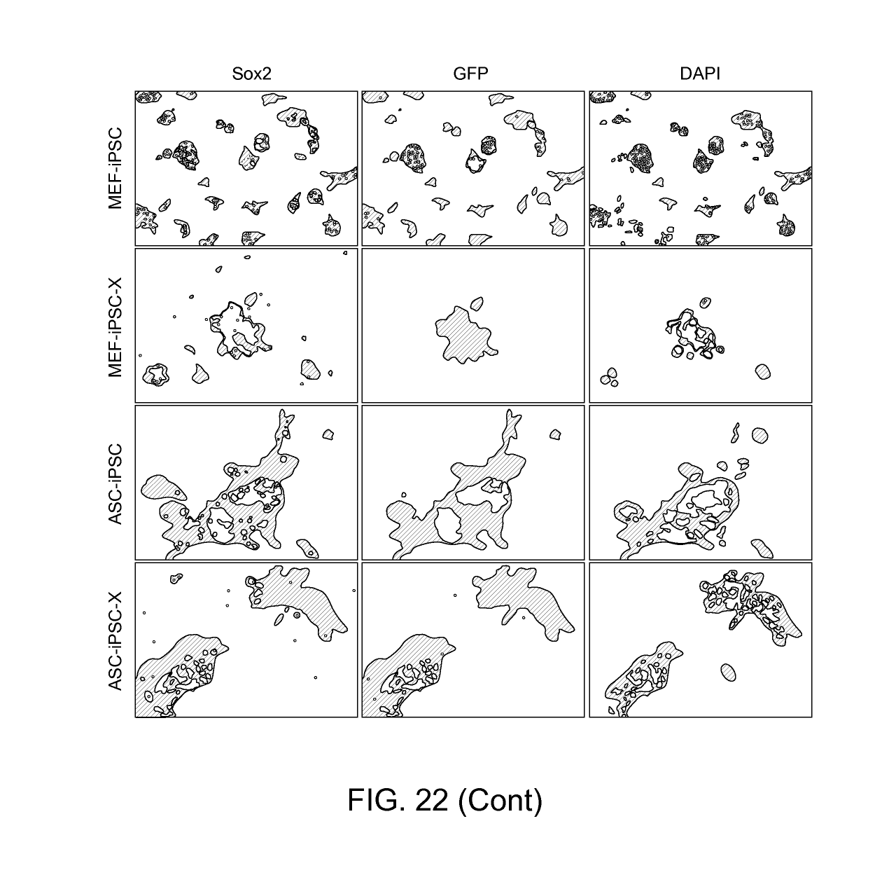

Immunofluorescence staining for the ESC/iPSC-characteristic proteins Nanog, and Sox2 revealed their expression (FIG. 22).

### Example 1

### PhiC31-Mediated Integration of Dystrophin Gene in Mammalian Cells

FIG. 1 provides a schematic diagram of φC31-mediated integration of dystrophin gene in mammalian cells.

φC31 integrase. φC31 integrase is a sequence-specific recombinase encoded by a phage of Streptomyces soil bacteria. The enzyme performs efficient and precise recombination between two short sequences, called attachment sites or attB and attP sites, for the purpose of inserting the phage genome into the host chromosome. φC31 integrase also works in mammalian cells (Groth, A. C., Olivares, E. C., Thyagaraj an, B., and Calos, M. P. 2000. Proc Natl Acad Sci USA 97:5995-6000) and can efficiently insert a plasmid carrying an attB site into mammalian genomes at native sequences called pseudo attP sites that resemble the attP site (FIG. 1). Two plasmids, one encoding dystrophin and attB and the other encoding integrase, were transfected into cells. Integrase was encoded and pairs the attB site on the plasmid with a pseudo attP site in the chromosome, bringing about permanent integration of the dystrophin at an endogenous attP site in the chromosome.

The φC31 integrase system is a simple plasmid DNA approach, comprising co-transfection into target cells of a plasmid carrying the attB site and the therapeutic gene, along with a plasmid expressing the integrase. There is no viral vector involved, which eliminates problems associated with viral immunogenicity and toxicity and makes the φC31 integrase system safe to use and inexpensive to manufacture.

Because the φC31 integrase system requires a DNA sequence match with the genome in order to integrate, it uses a much more limited number of integration sites compared to other DNA integration vectors such as transposons and retroviruses, which integrate essentially at random. This is an important safety feature, because random integration can lead to activation of oncogenes.

The φC31 integrase system lacks a size limit, so genes of any size, complete with control regions, can be integrated. For example, φC31 integrase has been used to integrate large plasmids of over 100 kb. This lack of size limit is of particular relevance in treatment of DMD, because the full-length dystrophin cDNA is ˜14 kb long (Koenig, M., et al., 1988, Cell 53:219-228.). Other gene transfer methods such as adeno-associated virus and lentiviral vectors are unable to carry the full-length dystrophin cDNA. The φC31 integrase system has been proven to transfer the full-length dystrophin cDNA in several studies (Bertoni, C., et al., 2006, Proc Natl Acad Sci USA 103:419-424; Quenneville, S. P., et al., 2004, Mol Ther 10:679-687; Quenneville, S. P., et al., 2007, Gene Ther 14:514-522).

The safety of using integration sites used by φC31 integrase in human cells is rigorously examined in the examples presented below.

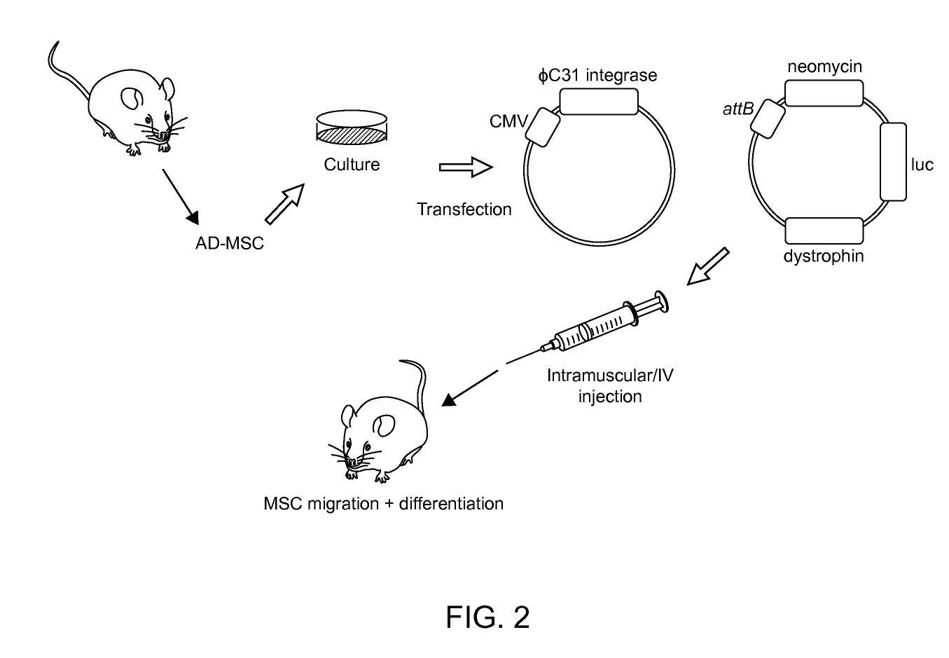

The strategy for correcting dystrophin deficiency in stem cells derived from mouse models is outlined in FIG. 2.

### Example 2

### Isolation and Characterization of Adipose Derived-Mesenchymal Stem Cells

A source of autologous multipotent, myogenic cells that are available in large numbers and can be easily accessed from patients would be desirable. Adipose-derived mesenchymal stem cells (AD-MSCs) appeared to represent such cells, so experiments to isolate and characterize mouse and human AD-MSC were performed.

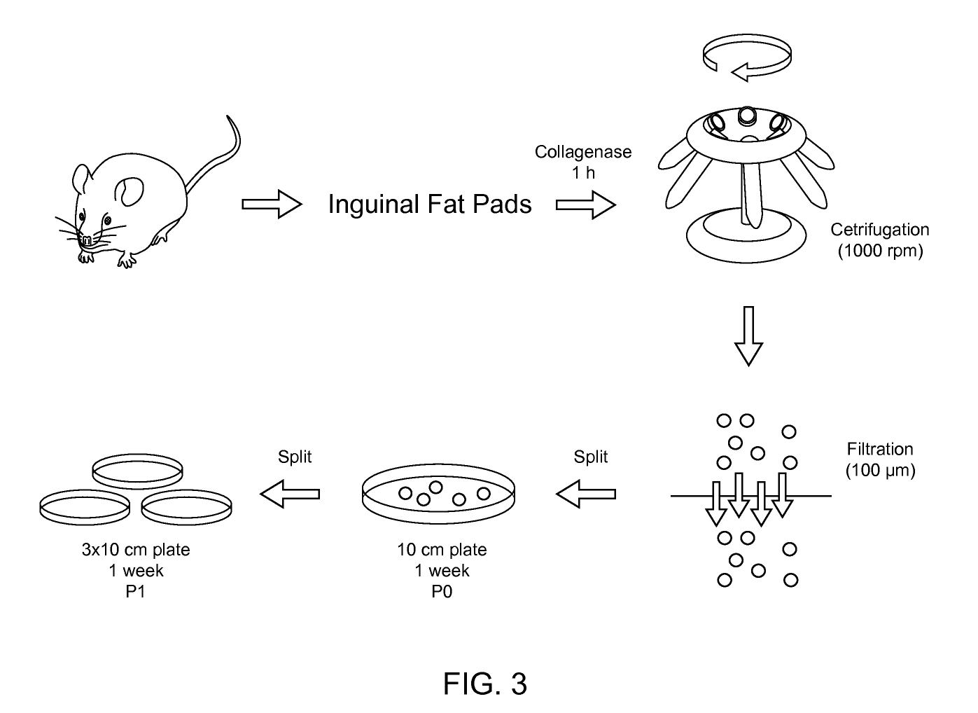

Mouse AD-MSCs were isolated from inguinal fat pads. The isolation of AD-MSC is described in FIG. 3 and is essentially a one-day procedure. Feeder cells are not used.

FIG. 3 provides a schematic of the procedure for isolation and culture of AD-MSCs. Fat pads were rinsed & minced in HBSS. Adipose tissue was digested with 0.075% collagenase type IV, 37° C., 1 h. Collagenase was deactivated with DMEM supplemented w 10% FBS. Cells suspensions were centrifuged @ 1000 rpm to remove adipocytes. Resulting cell pellets were resuspended in DMEM supplemented with 10% FBS and passed through 100 mm sterile filters. AD-MSCs were cultured in this medium, with media changed every 2 days.

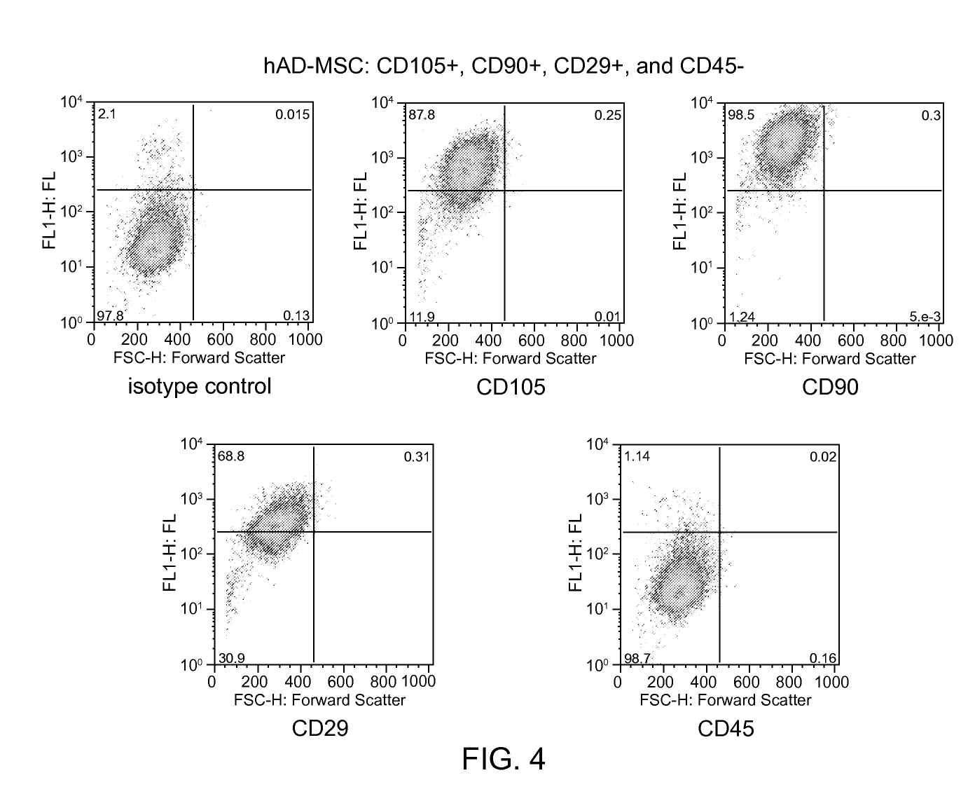

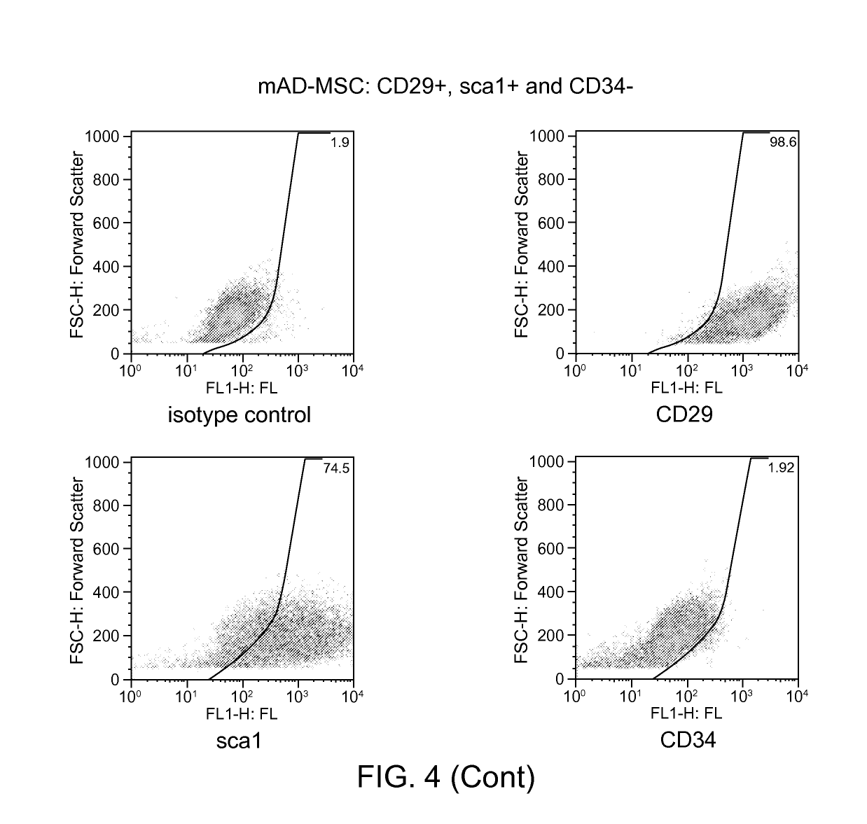

FACS analysis for MSC cell surface markers. In order to verify that the cells isolated in this procedure were MSCs, the cells were analyzed for surface markers characteristic of MSCs. As shown in FIG. 4, the majority of cells in the AD-MSC preparation from human samples (hAD-MSC) was positive for CD105, CD 90, and CD 29, and was negative for CD 45. FACS analysis of the mouse AD-MSCs (mAD-MSC) indicated that most of the cells expressed CD29 and Scat and did not express CD34 (FIG. 4). These patterns of cell surface marker expression are typical of MSC. Therefore, these preparations consisted substantially of MSC.

FIG. 4 depicts analysis of AD-MSC surface marker expression. AD-MSC were stained with FITC-conjugated antibodies and analyzed via flow cytometry.

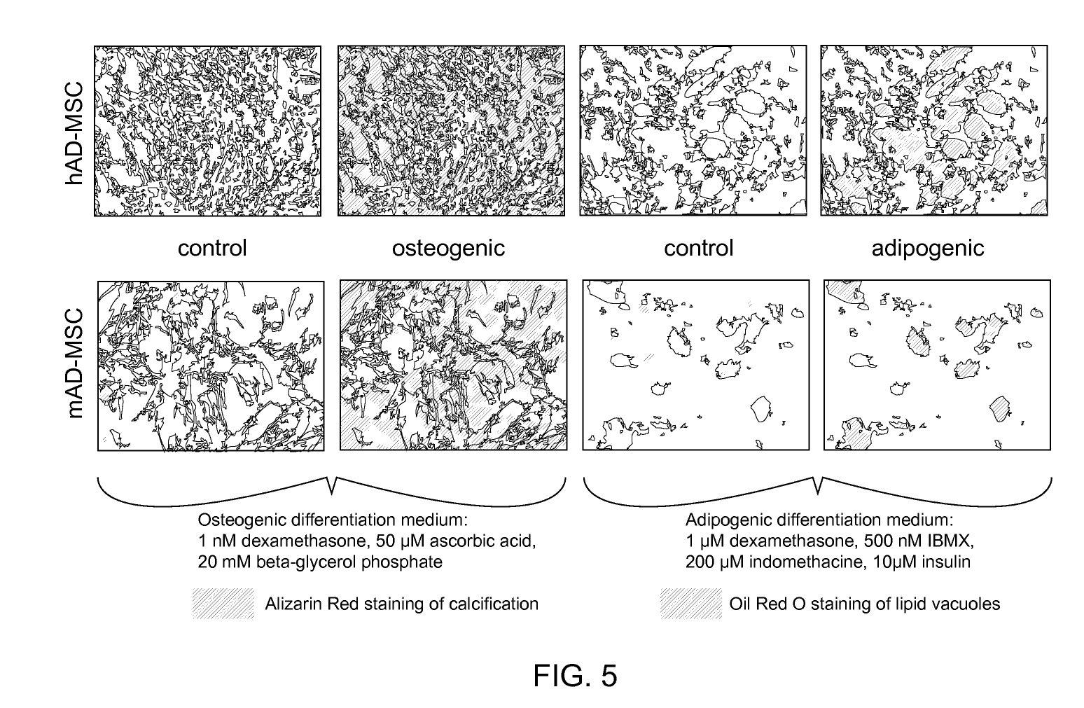

In vitro differentiation of AD-MSC. Human and mouse AD-MSCs were characterized to verify that they had appropriate in vitro differentiation capacities, based on prior studies of in vitro differentiation of MSC into various types of mesenchymal cells such as osteoblasts and adipocytes. As shown in FIG. 5, the human and mouse AD-MSCs readily differentiated along the osteogenic and adipogenic lineages after two weeks of culture in the appropriate differentiation media.

FIG. 5 illustrates differentiation of AD-MSC. AD-MSCs were differentiated into the osteogenic and adipogenic lineages. After 2 weeks, cells were stained for calcification (alizarin red staining) and lipid vacuoles (Oil Red 0 staining), respectively. Osteogenic differentiation medium included 1 nM dexamethasone, 50 μM ascorbic acid, 20 mM beta-glycerol phosphate.

### Example 3

### Transfection of AD-MSCs

Transfection conditions for the mouse and human AD-MSCs were optimized by testing various transfection methods and monitoring short-term GFP fluorescence after transfection of a plasmid carrying the eGFP gene and attB. Transfection reagents were used according to the manufacturers' directions, including varying amounts of DNA. Two wells of a 6-well plate were transfected with the pDB2 plasmid (carrying eGFP and attB) using each reagent. FACS analysis was done after 72 hours to determine the percentage of cells that were alive and GFP-positive. Cells were stained with a solution of 5 mg/ml propidium iodide in phosphate-buffered saline to mark dead cells. Transfection reagents included Effectene (Qiagen), SuperFect (Qiagen), Lipofectamine 2000 (Invitrogen), Lipofectamine Plus (Invitrogen), FuGENE 6 (Roche), and FuGENE HD (Roche), and electroporation with the Amaxa nucleofector. Cells were analyzed on a FACScan machine (BD Biosciences). Amaxa nucleofection resulted in the highest transfection efficiency, at about 25-45% (FIG. 4). When a plasmid expressing phiC31 integrase was co-nucleofected and genomic DNA was prepared from the cells several days after transfection, a PCR band indicating genomic integration of the GFP-attB plasmid at the most common phiC31 integration site in the mouse genome, mpsL1 was detected. These results indicated that: 1) the attB and phiC31 integrase plasmids could be co-transfected into mAD-MSC at a reasonable frequency, and 2) the expected sequence-specific integration into the mouse genome occurred.

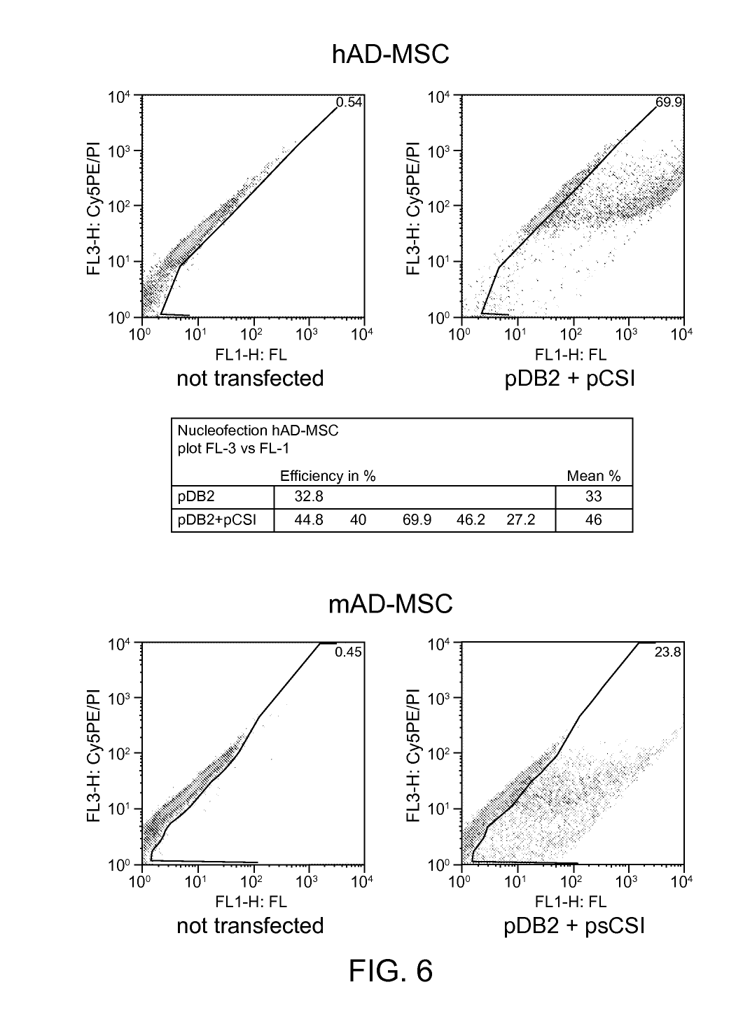

FIG. 6 illustrates nucleofection of AD-MSCs. AD-MSCs were nucleofected (Amaxa) with a GFP reporter construct (pDB2) together with phiC31 integrase (PCSI) and analyzed via flow cytometry 72 hours after nucleofection. 3 μg total DNA (Ratio 1:1), 4−5×10⁵.

### Example 4

### Engraftment of MD-MSCs into Muscle

AD-MSCs may possess sufficient migration and differentiation properties critical for creating a beneficial therapy for DMD. A luciferase/GFP transgenic mouse will be used as a tissue donor for quantitative evaluation and optimization of engraftment.

The transgenes are transcribed from the CAG promoter and are constitutively expressed in all tissues except mature erythrocytes. These mice are available from Jackson Laboratory and the Contag lab (Stanford University). The mice are on the FVB strain background and are thus incompatible with mdx. Therefore, donor cells will be transplanted into unlabelled FVB recipient mice that have been cryoinjured or cardiotoxin-treated to provide a degenerating muscle environment.

For intramuscular injection, cells will be counted on a haemocytometer and resuspended in HBSS buffer (Invitrogen) at a concentration of one million cells per ˜60 μl. For injection into the TA muscle, mice are given an intraperitoneal injection of 150 mg/kg ketamine and 10 mg/kg xylazine and the injection site is shaved. A 1 cm-long cut is made to the skin to visualize the TA muscle, which is sutured after injection. One million cells are injected per mouse.

Live in vivo imaging of luciferase expression is a quick and non-invasive procedure that will allow the same mice to be imaged over a time course to observe directly the migration and engraftment of the cells. Briefly, anesthetized mice will be given an intraperitoneal injection with luciferin substrate (150 mg/kg), placed inside the imaging box, and imaged by a CCD camera. The whole procedure takes 20-40 minutes for a mouse and provides an immediate quantitative readout of the extent and distribution of luciferase expression. The mice injected with AD-MSC can be imaged starting as early as 24 hours after injection. Initial luciferase levels are expected to be at least two orders of magnitude higher in injected versus untreated muscles, based on our preliminary studies.

Treated and untreated mice will be imaged the day after injection and weekly afterwards to track the incorporation of the AD-MSC into skeletal muscle and the continued expression of the integrated luciferase gene. At various time points after the last injection of AD-MSC, mice will be euthanized. Skeletal muscle will be dissected and examined for expression of GFP by fluorescence, Western blot, reconstitution of GFP-positive muscle fibers by immunohistochemistry, and presence of the luciferase and GFP genes by PCR.

Engraftment to levels >25% in skeletal muscles can be achieved by performing additional injections of cells. In addition to IM route of injection of AD-MSC, systemic injection by tail vein or intra-arterial can also be employed. In the case of systemic injection, cells should be free of clumps to reduce the risk of thrombosis. Migration ability of MSC can be improved by exposure to cytokines in vitro. Addition of genes specifically to enhance survival of transplanted cells, such as VEGF, or treating the cells with Matrigel to enhance engraftment, may also be used. Addition of the MyoD gene under control of a weak constitutive promoter (PGK) greatly increase the ability of hAD-MSC to engraft in cryoinjured immunedeficient mice. This strategy could be employed by including the MyoD expression cassette on the therapeutic plasmid.

### Example 5

### Use of Mouse AD-MSC Corrected with φC31 Integrase to Treat mdx5CV Mice

Mouse AD-MSC, in combination with the φC31 integrase system to correct autologous cells genetically, can be used to create an effective therapy for the mdx^{5CV} mouse model of DMD.

Plasmids encoding the full-length mouse or human dystrophin cDNAs will be used. The muscle-specific creatine kinase 6 (CK6) promoter will drive the dystrophin cDNA to provide sufficient expression. A strain of mdx mice, commonly called mdx^{5CV}(Dmd^{mdx-5CV} available from the Jackson Laboratory), that display 10 times fewer dystrophin-positive revertant fibers than other mdx strains, will be used in these experiments in order to monitor engraftment with more sensitivity. These mice can be obtained from the laboratory of Dr. Thomas A. Rando.

AD-MSC will be isolated from mdx^{5CV} mice and co-transfected with two plasmids. One plasmid encodes the φC31 integrase, which will direct integration of the attB-containing plasmid. The other plasmid carries an attB site to allow the integration of the plasmid into the genome by the φC31 integrase. The attB-donor plasmid also carries the neomycin resistance gene and luciferase gene. The neomycin resistance gene will allow for a quick drug selection in vitro so that only cells carrying the integrated attB-donor plasmid will survive. This step will eliminate cells that were not transfected or did not have an integration event.

After using the optimal transfection conditions established in the previous example (see FIG. 6) to introduce the φC31 integrase and dystrophin-neo-attB plasmids, neomycin-resistant cells will be selected. A population of transfected cells will generally contain cells that have φC31 integrase-mediated integrations at several different locations, with one integration event per cell. The level of dystrophin expression will be monitored by Western blot, using antibodies against dystrophin purchased from Santa Cruz Biotechnology. Mock-transfected cells will be used as a negative control.

For intramuscular injection, one million cells in ˜60 μl will be injected into the TA muscle. At various time points after the last injection of AD-MSC, mice will be euthanized. Skeletal muscle will be dissected and examined for expression of dystrophin by Western blot, reconstitution of dystrophin-positive muscle fibers by immunohistochemistry, and integration of the dystrophin plasmid at the mpsL1 site by PCR. Using the conditions established in example 4 to maximize engraftment to levels >25%, positive results in the Western blots for dystrophin s expected. Moreover, a significant fraction of dystrophin-positive fibers by immunofluorescence for dystrophin is predicted to be detected, especially compared to the low background of dystrophin expression in the mdx^{5CV} mice. Once substantial engraftment levels are obtained, muscle strength will be measured in order to document functional improvement as a result of the therapy.

### Example 6

### Characterization and Safety of Human AD-MSC Corrected with φC31 Integrase

Human AD-MSC will be subjected to transfection and phiC31-mediated integration of a plasmid encoding human dystrophin-HA cDNA, and safety studies will be carried out with the cells.

Human AD-MSC derived from normal subjects undergoing liposuction procedures will be used. Transfection conditions for hAD-MSC (see FIG. 6) will be used to co-transfect cells with two plasmids, one encoding the φC31 integrase and the other carrying attB, the neomycin resistance gene, and the human dystrophin cDNA, tagged with the 8 amino acid hemagglutinin epitope (HA) for detection of transgene expression. The HA tag will allow distinguishing of dystrophin made by the transgene from the endogenous dystrophin. (Because the hAD-MSC are obtained from unaffected donors, they are expected to synthesize wild-type dystrophin upon differentiation.) Populations of neomycin-resistant cells having integration events will be propagated. Genomic DNA from these cells will be analyzed. Presence of the introduced human cDNA will be distinguishable by PCR, due to its lack of introns. The ability of these cells to differentiate upon co-culture with myoblasts will be investigated, as outlined above. Expression of the dystrophin transgene will be analyzed by Western blot and by immunohistochemistry, using an antibody against the HA tag. The HA antibody works well and should have no cross-reactivity with endogenous dystrophin.

Integration sites will be analyzed initially by using previously developed panel of PCR primers for detection of the eleven most frequently used φC31 integration sites in the human genome (Chalberg, T. W., et al., 2006, J Mol Biol 357:28-48.). This analysis will verify that sequence-specific φC31-mediated integration has occurred and will provide a preliminary profile of the genomic locations at which integration takes place in hAD-MSC. A more comprehensive analysis of the DNA sequences of integration sites will be determined by using 454 pyrosequencing. This recently-developed sequencing method has been used, for example, to determine the DNA sequences of large numbers of lentiviral integration sites (Wang, G., et al., 2007, Genome Res 17:1186-1194). To apply the method, ligation-mediated PCR will be used to amplify all fragments containing one end within the integrated plasmid and the other end in neighboring genomic sequence. This material will be sequenced with the 454 device (Roche), and resulting sequences will be subject to BLAST analysis to determine genomic location of integration. This data will be compared to a comprehensive list of genes associated with cancer, to determine whether any of the integration sites are in proximity to known cancer genes.

To further query the safety of the hAD-MSC with integrated dystrophin plasmid, karyotype analysis on the cells will be performed before and after integration. Chromosome numbers and whether chromosome rearrangements are present will be determined. To test the safety of the cells biologically, they will injected subcutaneously in SCID mice and monitored over a long time course for tumor formation.

### Example 7

### Reprogramming and Genetic Correction of AD-MSC

A population of patient-specific stem cells that have been reprogrammed, gene-corrected, and differentiated into muscle precursor cells for engraftment will be produced. Adipose-derived mesenchymal stem cells (AD-MSC) can be used for this purpose, since they are simple to obtain, are easily purified and cultured, and are efficiently reprogrammed. An elegant 3-step, non-viral strategy for genetic engineering of the cells will be used. The strategy makes use of sequence-specific recombinases for precise engineering of mammalian genomes (FIG. 7).

Cells are efficiently reprogrammed into induced pluripotent stem cells (iPSC) by using a sequence-specific phage integrase to insert the reprogramming genes into one safe site. After reprogramming, the transcription factor genes are removed by transient exposure of the cells to Cre recombinase, leaving behind a landing pad for a second integrase, called R4. A plasmid carrying the full-length dystrophin cDNA is then added efficiently and site-specifically at this location by R4 integrase. The gene-corrected population of iPS cells is differentiated in culture toward the myogenic lineage, so that it is enriched in muscle progenitor cells. These muscle precursor cells are collected by FACS sorting and used for engraftment. The efficacy endpoint is increased muscle strength.

The muscle degeneration that is ongoing in DMD may be best addressed by a stem cell intervention to replace lost muscle fibers. Target product cells have myogenic capacity, as shown by in vitro differentiation and in vivo studies in animal models. Cells can engraft and fuse, forming functional muscle fibers in diseased muscles. The cell preparation has been sorted for muscle-specific cell surface markers, to enrich the activity of the preparation and to exclude undifferentiated cells that could give rise to teratomas. Cell delivery is by intramuscular injection, which is safe and provides local engraftment. In addition, systemic injection may lead to more widespread distribution of cells, which is useful for reaching critical targets such as the diaphragm. Owing to an anticipated lack of immunological bathers, a regimen of sequential administration of therapeutic cells is envisioned, to extend the range of engraftment and regeneration. The proposed experiments will develop the complete strategy in disease model mice, using both mouse and human cells.

The target product has the desired therapeutic qualities. It is a muscle precursor cell similar to the satellite cells that ordinarily give rise to new muscle fibers during normal growth and muscle repair. The target produce is derived from the patient and therefore can be transplanted without immunological rejection. Since the target product cell is corrected for dystrophin, it synthesizes appropriate quantities of intact dystrophin protein. The dystrophin protein will become localized to the cell membrane where it will function to preserve the muscle fiber. Muscle fibers are formed by fusion of progenitor myoblasts to form a long, multinucleated fiber that is a syncytium of many cells. Upon fusion with existing muscle fibers, the dystrophin made by the target product will distribute along the fiber and protect the entire fiber. The target product has the capacity to generate new muscle fibers over the long-term that will synthesize dystrophin and be functional.

The target population for the therapy is all individuals with DMD. Treatment of young children is likely to be most effective, since the target product cells can became incorporated into muscles before extensive degeneration has occurred. However, the therapy is expected to be beneficial at any stage of the disease. Intramuscular delivery will distribute the target product to affected muscles and improve quality of life, while systemic delivery has the potential to reach the diaphragm and heart, which must be targeted to achieve a normal lifespan. Success criteria involve successful incorporation of dystrophin positive muscle fibers into patient muscles. A high substitution rate is desirable, manifesting as more healthy muscle fibers, increased muscular strength, improved mobility, and, if the diaphragm and heart can be reached, increased lifespan.

### Example 8

### Reprogramming of AD-MSCS to Form iPSCs

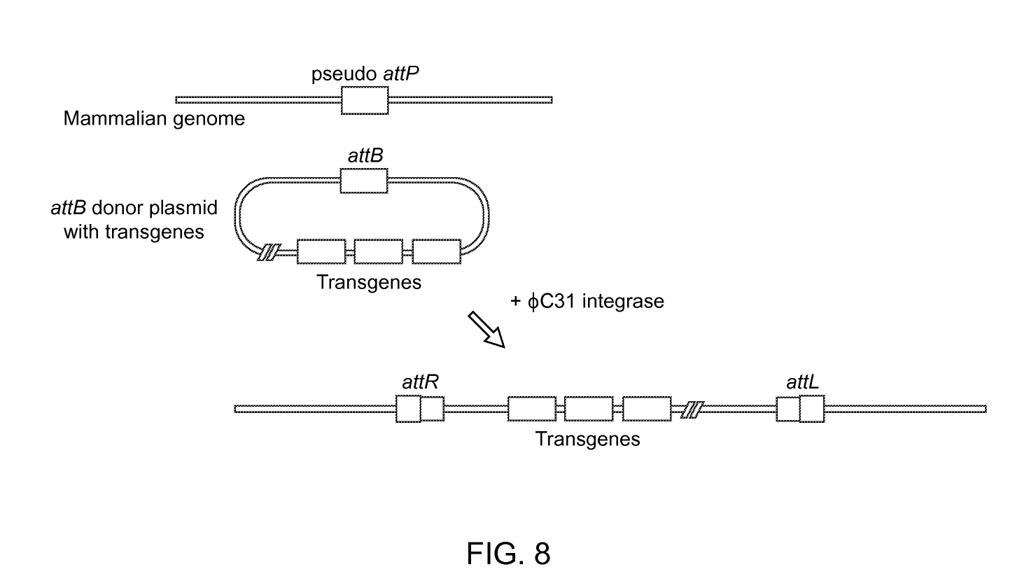

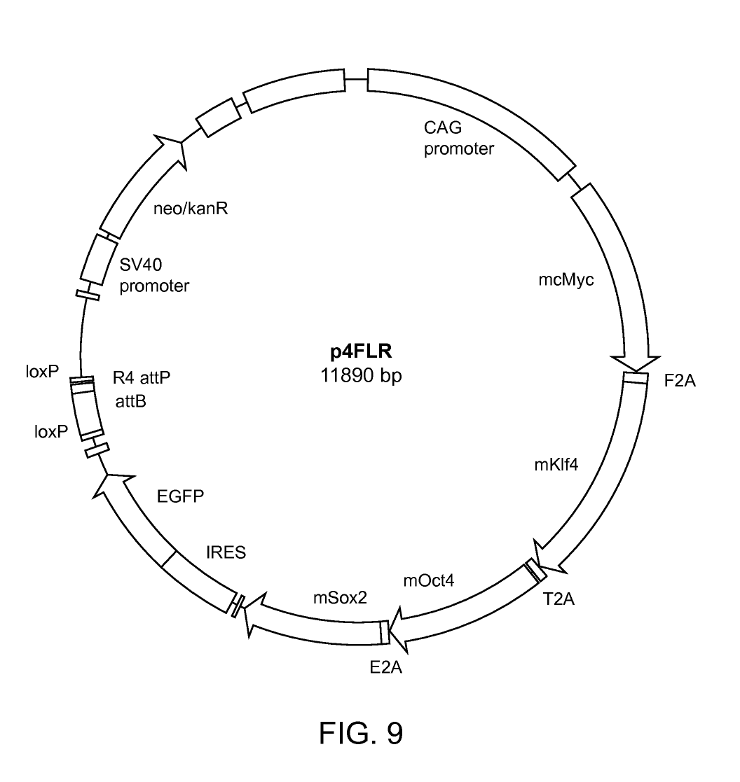

For making iPS cells with φC31 integrase, reprogramming genes were cloned into an attB donor plasmid, which would result in integration at one site (FIG. 8). Introduction of reprogramming plasmid p4FLR (FIG. 9) into mouse embryo fibroblasts or AD-MSC by nucleofection, along with a plasmid encoding φC31 integrase, led to iPSC colonies by 10 days, at a frequency similar to that obtained with retroviruses. A similar plasmid was used to obtain iPSC from adult human AD-MSC, taking advantage of reprogramming cassettes previously used to reprogram human cells effectively (Jia, F., et al., 2010, Nature Methods 7:197-199.).

The pluripotency of the iPS cells obtained with this strategy was validated. The iPS cells generated by φC31 are indistinguishable from ES cells by the criteria of alkaline phosphatase staining, immunofluorescence staining for Oct4, Sox2, Nanog, and SSEA1, and embryoid body differentiation into the three germ layers, as verified by staining for ectoderm with β-III tubulin to detect neural cells, mesoderm with smooth muscle actin, and endoderm with α-fetoprotein. qRT-PCR was used to demonstrate the reactivation of endogenous transcription factor expression including Oct, Sox, and Nanog, while karyotyping of the iPS lines revealed that most have a normal karyotype.

### Example 9

### Excising Reprogramming Genes from iPSCs and Introducing Dystrophin cDNA

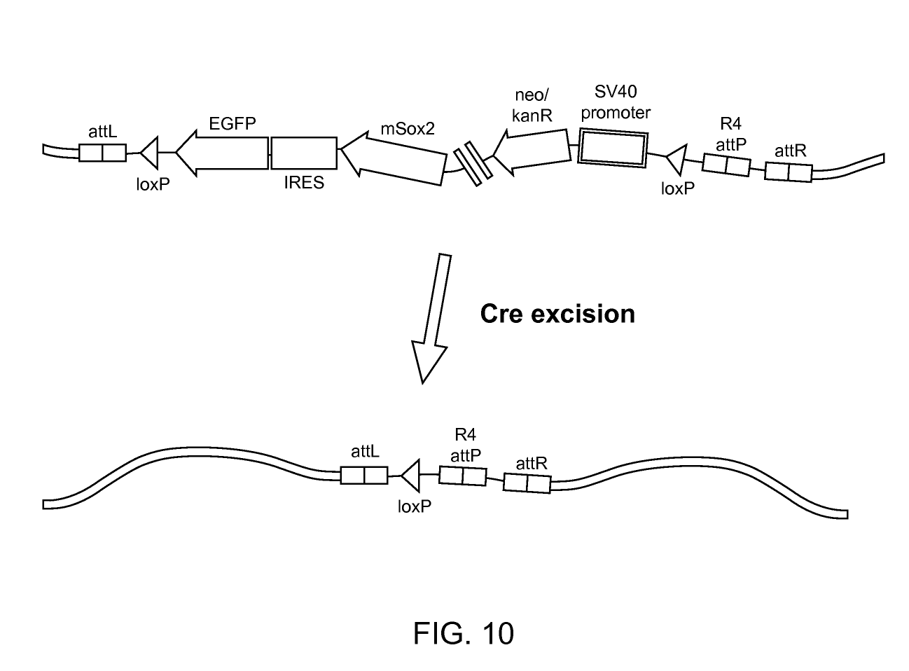

Two additional features to the recombinase strategy were added. In addition to the φC31 attB site for integration of the reprogramming plasmid into the mammalian genome with φC31 integrase, the reprogramming plasmid also contains recombinase recognition sites for Cre and for R4 integrase. These sites allow the deletion of the reprogramming genes after an iPS cell has been formed and then to add a therapeutic gene at the same position. While recombinase recognition sites are small, about 34 base pairs in length, they are long enough to be unique in the mammalian genome. The sites are recognized strongly by their cognate recombinases, which carry out precise, sequence-specific recombination reactions at these sites (FIG. 10).

FIG. 10 shows details of arrangement of recombinase sites from p4FLR after genomic integration of a reprogramming cassette. Integration occurred via the φC31 attB site, which recombined with a genomic pseudo attP site, yielding inactive hybrid sites known as attL and attR. The reprogramming genes were flanked by loxP sites so that transient exposure of the cells to Cre recombinase led to clean excision of the genes. Left behind was the attP site of R4, ready for precise integration of the dystrophin gene.

Once an iPSC is formed, there is generally no further requirement for the reprogramming genes. Precise excision of the genes is a simple matter in these cells, since there is just one integration site. Transient exposure of the cells to Cre recombinase achieved efficient removal of the reprogramming cassette in most cells. Cre exposure was achieved either by nucleofection of a plasmid encoding Cre, such as pCAG-Cre or by addition of TAT-Cre protein to the medium. This form of Cre fused to a cell-penetrating TAT peptide also produced efficient excision. In this case, the cells were spared an additional round of electroporation, and there was no chance of integration of the recombinase plasmid. Both Cre plasmids were obtained from Addgene. Excised cells were readily identified by loss of GFP fluorescence.

The R4 attP site was then used to target integration of a plasmid carrying the full-length dystrophin cDNA and the R4 attB site (FIG. 7). When the R4 attP site is placed into a favorable genomic position by φC31 integrase, it attracts precise integration with 95% specificity and at a 25-fold higher integration efficiency than genomic integration into a pseudo attP site (Olivares, E. C., et al., 2001, Gene 278:167-176). Therefore, it was a simple matter to identify cells that have the therapeutic gene integrated at the correct site. The karyotype and integration site had been previously analyzed for safety. The engineered iPS cells may then be expanded and differentiated down the muscle cell lineage.

A similar strategy may be used to generate and engineer human iPS cells from DMD patients.

### Example 10

### Differentiation of iPSCs

Differentiation conditions can be based on published protocols for human and mouse ES cells and mouse iPS cells. The best protocol can be developed through experimentation. The feasibility of differentiating engraftable muscle precursor cells from pluripotent stem cells was first demonstrated by Barberi et al in 2007 (Nature Medicine 13:642-648). Starting with human ES cells, this group used selective culture conditions and FACS sorting to purify skeletal myoblasts. These cells could undergo in vitro differentiation into twitching myotubes and engrafted after transplantion into the tibialis anterior (TA) muscle of SCID mice, without teratoma formation.

The Perlingeiro group reported an alternative protocol in 2008, in which an inducible Pax3 gene was integrated into mouse ES cells (Darabi, R., et al., 2008, Nature Medicine 14:134-143). The Pax3 transcription factor is involved in normal initiation of the myogenic program within early paraxial mesoderm during development. Expression of Pax3 greatly enriched the myogenic potential of the cells. By sorting for the PDGF-α receptor, a marker of early mesoderm, and for the absence of FLK-1, a marker of late mesoderm, a population of cells was derived that gave rise to muscle fibers in vitro and in vivo, including studies in mdx mice, without the formation of teratomas. This protocol thus represents an interesting option, although derivation of abundant myogenic cells without addition of Pax3 has also been demonstrated.

A third group showed that, after appropriate culture conditions, mouse ES cells enriched for Pax7-positive satellite-like cells could be isolated by FACS sorting with a novel anti-satellite cell antibody, SM/C-2.6 (Chang, H., et al. 2009. FASEB Journal 23:1907-1919.). Pax7 is a transcription factor essential for satellite cell formation. Satellite cells are muscle stem cells that can both self-renew and differentiate into myoblasts and myotubes to form muscle fibers. The sorted cells efficiently differentiated into muscle fibers in vitro and in vivo after transplantation into mdx mice, with engraftment efficiency higher than that of myoblasts.

Furthermore, the engrafted cells continued to provide self-renewal in the engrafted muscle over many months, even after re-injury and secondary transplantation. These characteristics are very attractive for a therapeutic cell therapy for DMD. Very recently, the same group has obtained similar results when the starting cells were iPS cells rather than ES cells (Mizuno, Y., et al. 2010, Generation of skeletal muscle stem/progenitor cells from murine induced pluripotent stem cells, FASEB Journal.).

### Example 11

### Engraftment of Differentiated Cells

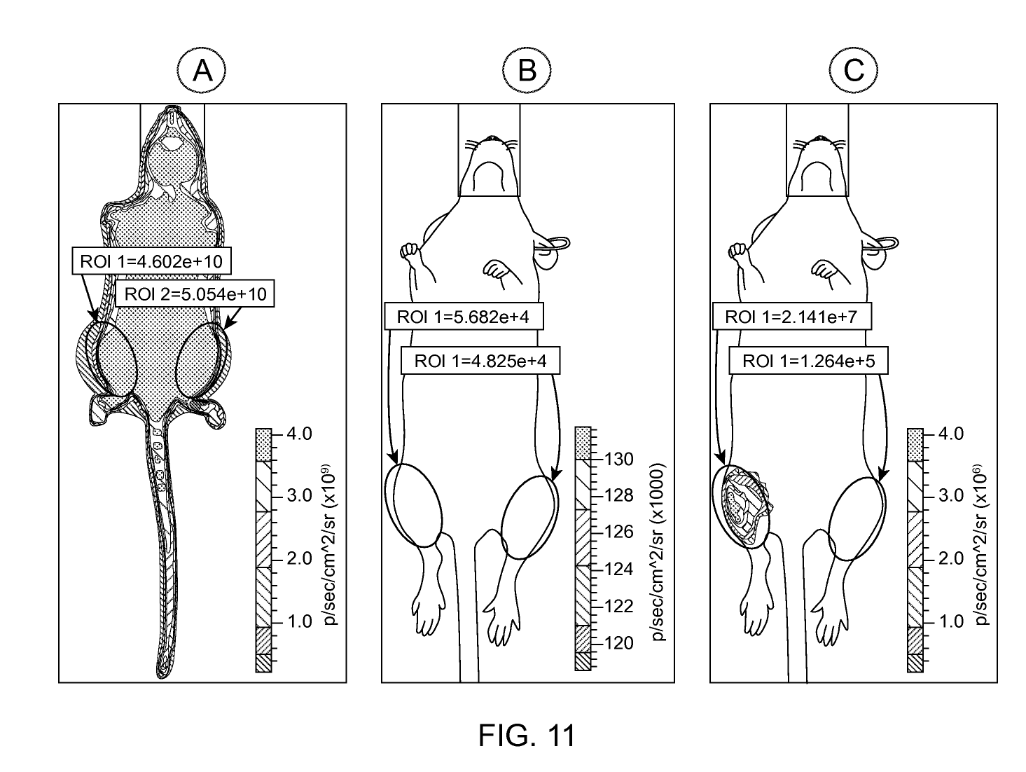

Use of transgenic mice expressing luciferase and GFP as cell donors for engraftment experiments is described in Example 4. FIG. 11 illustrates their efficacy for monitoring muscle engraftment.

FIG. 11 shows tracking of engraftment with luciferase live imaging. Panel A. Donor luciferse-GFP mouse shows strong labeling throughout the body. A 9-week old L2G85 heterozygous transgenic mouse (exposure time=0.5 s); Panel B. Unlabelled recipient mouse that received buffer alone shows background levels of fluorescence. A 14-week old FVB female injected in the right tibialis anterior with HBSS (3 weeks after injection, exposure time=10 sec); Panel C. Recipient mouse injected with 500,000 myoblasts from the labeled donor shows labeling that is about 200-fold above background 3 weeks after injection, indicating effective engraftment. A 14-week old FVB female injected in the right tibialis anterior with myoblasts isolated from a heterozygous L2G85 heterozygous transgenic mouse (3 weeks after injection, exposure time=10 sec). Photons/second over the oval regions are shown in the figure.

iPS cells will be derived from the luciferase-GFP mice, the cells differentiated into muscle precursors as outlined in Example 10, and the labeled cells will be used for studies designed to optimize engraftment. These studies will include intramuscular injection, systemic injection, multiple doses, and other variations that have previously been successful to maximize distribution and engraftment of the cells. In addition to the live-imaging fluorescence signal, engraftment on the histological and molecular level will be quantified.

When suitable engraftment conditions are developed, leading to muscle engraftment of >10%, the dystrophin-positive engineered mdx iPS cells will be engrafted into mdx mice. In the presence of good levels of engraftment, studies to measure improvement in muscle strength will be carried out. For example, force measurements on isolated TA muscle and improved rotarod performance have been useful in this context.

Derivation of human iPS cells and their differentiation into muscle precursors can be guided by the mouse work. AD-MSC from normal subjects can be used in initial studies, in which the luciferse gene can be integrated to carry out engraftment optimization. Patient-derived cells can be required for studies in which the human dystrophin cDNA can be added. A large bank of cells from DMD patients is available from Coriell Institute. This Human Genetic Cell repository is sponsored by the U.S. National Institute of General Medical Sciences and consists of well-characterized cell lines from DMD patients of various ages, including untransformed fibroblasts suitable for reprogramming. Fresh tissue samples can be obtained from the Stanford DMD clinic.

### Example 12

### Generation of iPSCs by Using φC31 Integrase

The delivery of the reprogramming factors into either MEFs or ASCs was performed by conucleofection of plasmid pVI carrying the φC31 integrase gene and the reprogramming plasmid p4FLR (FIG. 14, Panel A). Plasmid p4FLR included cDNA sequences for the murine cMyc, Klf4, Oct4, and Sox2 genes under the control of the CAG promoter and connected via 2A peptides, facilitating polycistronic mRNA expression. To screen for stable integrants, the reporter gene EGFP was included in the reprogramming plasmid, linked via an internal ribosomal entry site or IRES at the 3′ end of the polycistronic mRNA gene product. Downstream of the EGFP gene was placed a cassette carrying recognition sites for three site-specific recombinases. The φC31 attB site was used for primary integration, while the R4 attP site provided for potential secondary integration. These att sites were flanked by two loxP sites to facilitate Cre-mediated removal of the reprogramming cassette (FIG. 14, Panel A). Nucleofection efficiencies, as judged by scoring of GFP+ cells by fluorescent activated cell sorting (FACS) analysis performed 48-72 hours after nucleofection, were in the range of 35%-64% (FIG. 18).

After 48 hours of nucleofection, cells were plated onto mitomycin C-treated MEF feeder layers and switched to ESC medium. The reprogramming efficiency was calculated by dividing the number of iPSC colonies on each plate that stained positive for alkaline phosphatase or SSEA1 (FIG. 14, Panel B, upper panel) by the number of cells plated on the respective plate. iPSC colonies were obtained from MEFs at an efficiency of 0.01%+/−0.006%, while iPSC colonies from ASCs occurred at 0.014%+/−0.009%. Factoring in the transfection efficiency, reprogramming efficiencies of approximately 0.03% were typically observed. After picking individual colonies 18-24 days after nucleofection, iPSC lines were established. These cell lines stained positive for alkaline phosphatase (FIG. 14, Panel B, lower panel) and were subsequently evaluated for the number of integration events via Southern blot analysis by using a probe directed against the EGFP reporter gene on the reprogramming plasmid (FIG. 14, Panel C).

Among 19 MEF-derived iPSC clones tested, 37% exhibited a single integration event, while of 13 ASC-derived iPSC clones, 31% exhibited single-copy integration of the plasmid. Overall, approximately 50% of the analyzed genomic DNA samples obtained from iPSC clones exhibited a double integration of the reprogramming plasmid, while the remaining 16% showed a triple integration. An overview of the different integration events among MEF-iPSC and ASC-iPSC is given in FIG. 19. For simplicity and as a proof of concept, two clones with a single integration site were used, one derived from MEFs and one from ASCs. To determine the chromosomal location of each integration site, the single integrants were subjected to linker-mediated (LM)-PCR. By using this method, the MEF-iPSC line was shown to possess a single integration into an intronic region of the Ptpn1 gene on chromosome 2. The ASC-iPSC line was found to have a single integration in an intergenic region on chromosome 1. The locations of both integration sites were verified via PCR of the genomic locus by using a combination of genomic and plasmid-binding primers, as depicted schematically in (FIG. 14, Panel D). Chromosome spreads of metaphase cells of the selected clones were analyzed and revealed the correct chromosome number and no major differences from mESCs (FIG. 20). However, more refined cytogenetic techniques would be required to reveal more subtle chromosomal rearrangements that may occur in iPSCs.

Of the 14 integration sites evaluated by LM-PCR, six clones were found in intergenic regions, six were located within an intron, and two sites were in an exon (FIG. 24). These results are similar to those obtained in a previous report and largely reflect the proportions of these elements in the genome, with some skewing toward genes. Integration sites obtained in this study were evaluated according to the criteria articulated in a recently published study (Papapetrou et al., Nat Biotechnol 2011; 29:73-78), which defined so-called genomic safe harbors. Of the six intergenic sites, two met the criteria proposed by this work, which represented 14% of all integration sites analyzed. The context of the integration sites is summarized in FIG. 24, in which the genomic safe sites are highlighted in gray. It has been determined that 23% of φC31 integration sites in the human genome are in safe locations.

### Example 13

### Deletion of Reprogramming Genes from iPSCs by Using Cre Recombinase

To remove the reprogramming cassette, the iPSC clones carrying one copy of the reprogramming plasmid were transiently exposed to Cre recombinase (FIG. 14, Panel A). Cre was introduced by lipofection with Effectene of a plasmid expressing Cre. By visually tracking the loss of EGFP expression, transgene-free iPSC clones were easily detected and picked for clonal expansion. Typically, 50% or more of the clones exhibited loss of EGFP expression. Excision of the reprogramming plasmid was verified by Southern blot (FIG. 14, Panel C). The Cre-mediated removal of the reprogramming cassette from the respective genomic loci was further demonstrated by PCR of the genomic locus using a combination of genomic and plasmid-binding primers (FIG. 14, Panel D). Moreover, the absence of the integrase-encoding plasmid pVI, which was used to integrate p4FLR, could be shown by PCR (FIG. 14, Panel D, lower panel). The excised clones were designated MEF-iPSC-X and ASC-iPSC-X.

### Example 14

### Pluripotency of iPSCs Before and After Cre-Mediated Excision

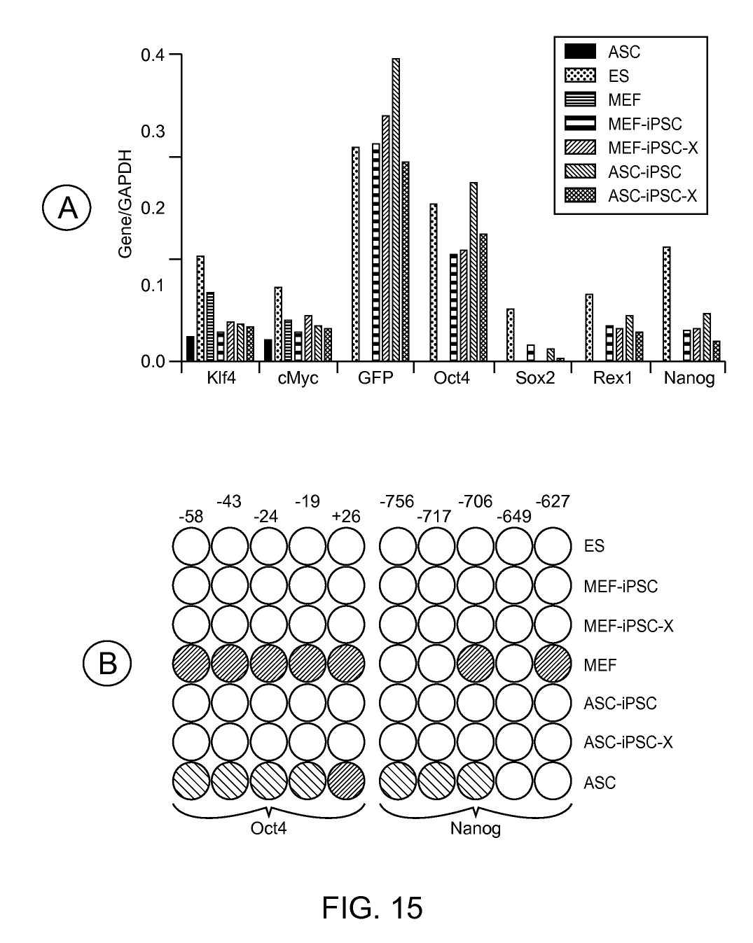

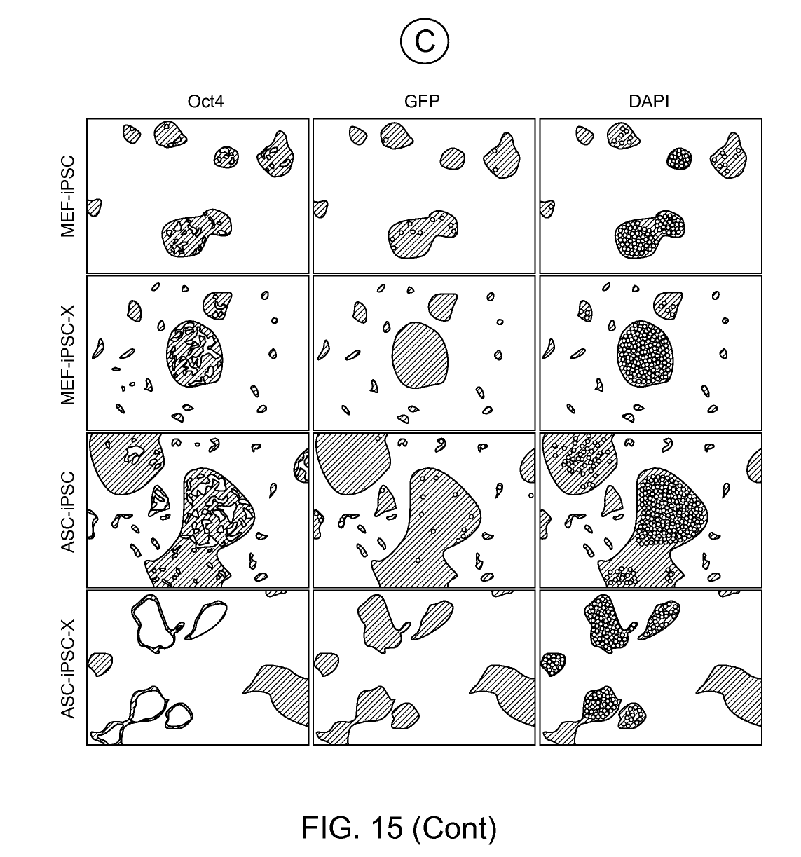

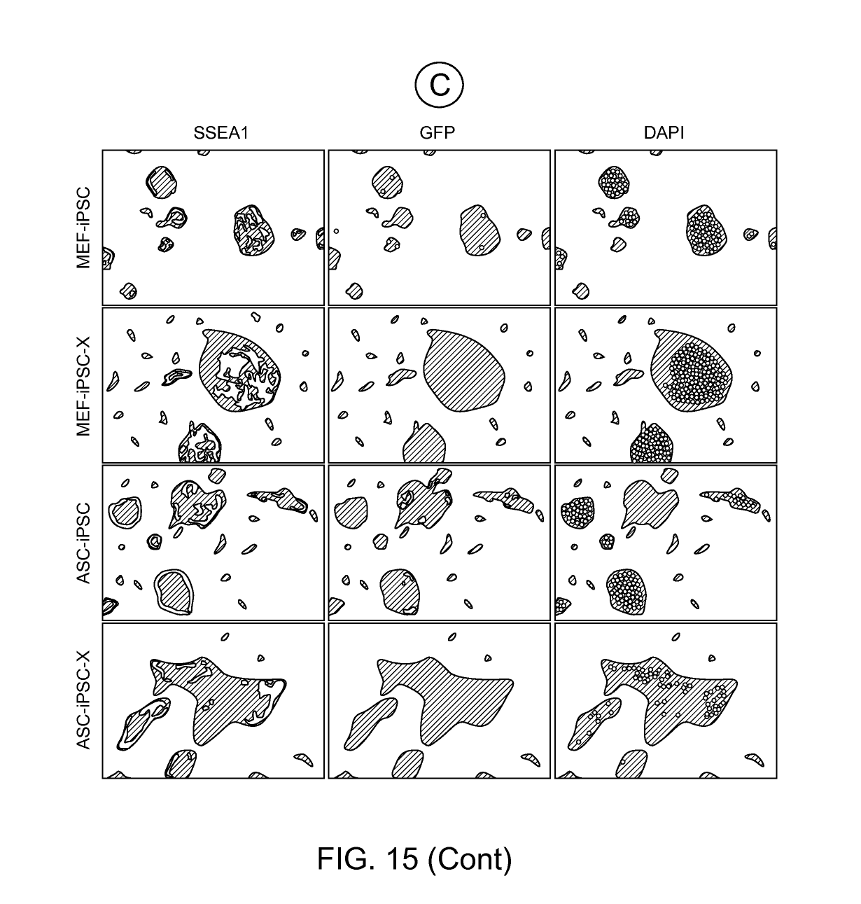

To evaluate the pluripotency of the iPSCs generated by using φC31 integrase, both before and after Cre-mediated excision of the reprogramming cassette, the following assays were carried out. The mRNA expression profiles of the pluripotency-associated genes Oct4, Klf4, Sox2, Nanog, and Rex1 as well as EGFP were determined via quantitative RT-PCR and compared with the respective transcript levels in mESCs. By comparing the transcript levels before and after removal of the ectopically expressed genes, reactivation of the endogenous gene transcripts was verified. As depicted in (FIG. 15, Panel A), the expression levels in the iPSC lines were similar to those in ESC.

To assess epigenetic changes in the DNA methylation status of the Oct4 and Nanog promoter regions, bisulfite sequencing was performed. Pyrosequencing revealed the full reactivation of the respective promoters, showing low methylation levels that were comparable with those of ESCs. In contrast, analysis of the promoter methylation in the parental MEFs and ASCs showed a high rate of methylation. FIG. 15, Panel B schematically depicts the results of the bisulfite pyrosequencing. The quantification can be seen in FIG. 21. Immunofluorescence staining for Oct4, SSEA1, Nanog, and Sox2 (FIG. 22 for the latter two markers) revealed expression of those ESCs/iPSCs-characteristic proteins (FIG. 15, Panel C). The removal of the reprogramming cassette, including the reporter gene EGFP, allowed validation of transgene-free iPSCs by the absence of EGFP staining. In the EGFP-negative MEF-iPSC-X and ASC-iPSC-X clones, sustained expression of the endogenous pluripotency-associated proteins was verified (FIG. 15, Panel C).

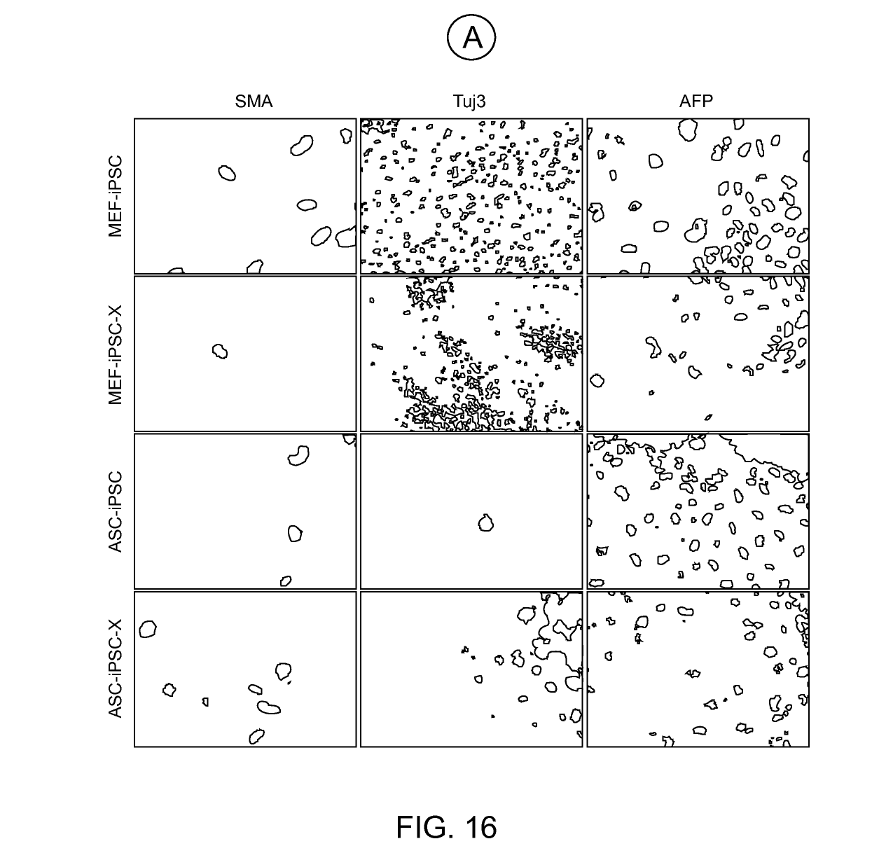

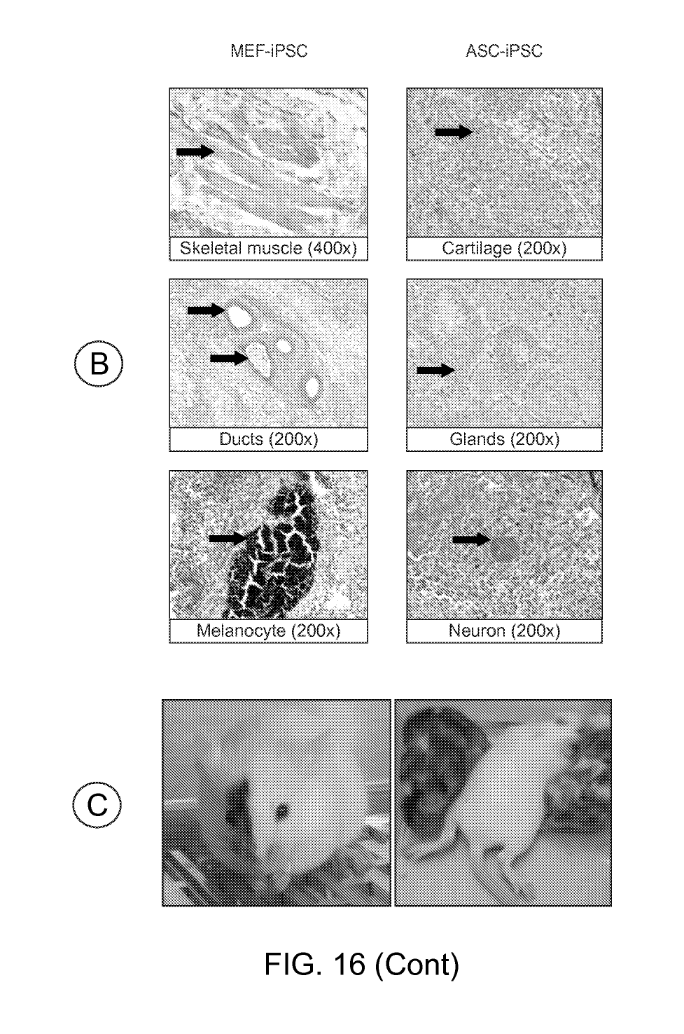

To assess the in vitro differentiation potential across all three germ layers of the iPSC clones, embryoid body formation was carried out. By staining for SMA, Tuj 1, and AFP, it was demonstrated that differentiation into cells of mesodermal, ectodermal, and endodermal origins, respectively, could be achieved (FIG. 16, Panel A). Furthermore, the pluripotency of the iPSC lines was not altered after removal of the reprogramming cassette, because the differentiation potential of MEF-iPSC-X and ASC-iPSC-X was not reduced (FIG. 16, Panel A).

To evaluate pluripotency in vivo, the iPSC clones were injected into the kidney capsule of immune-deficient SCID/beige mice, and teratoma formation was evaluated 4 weeks after injection. As shown in FIG. 16, Panel B for MEF-iPSC and ASC-iPSC, histological analysis of the teratoma revealed that cell types derived from all three germ layers were included in the tumors. The injection of MEF-iPSC-X and ASC-iPSC-X all led to teratoma formation to a similar extent (data not shown).

As a final proof of pluripotency, iPSCs were injected into the blastocysts of albino B6 mice and implanted into the uteri of pseudopregnant foster mothers. Contribution to chimeras was observed by patched coat color (FIG. 16, Panel C). Thus, the recombinase-generated iPSCs were genuinely reprogrammed, fulfilling all criteria of pluripotency.

### Example 15

### PhiC31-Mediated Integration of Dysferlin Gene in Mammalian Cells

φC31 integrase. φC31 integrase is a sequence-specific recombinase encoded by a phage of Streptomyces soil bacteria. The enzyme performs efficient and precise recombination between two short sequences, called attachment sites or attB and attP sites, for the purpose of inserting the phage genome into the host chromosome. φC31 integrase also works in mammalian cells (Groth, A. C., Olivares, E. C., Thyagarajan, B., and Calos, M. P. 2000. Proc Natl Acad Sci USA 97:5995-6000) and can efficiently insert a plasmid carrying an attB site into mammalian genomes at native sequences called pseudo attP sites that resemble the attP site (FIG. 1). Two plasmids, one encoding dysferlin and attB and the other encoding integrase, will be transfected into cells. Integrase is encoded and pairs the attB site on the plasmid with a pseudo attP site in the chromosome, bringing about permanent integration of the dysferlin gene at an endogenous attP site in the chromosome.

The φC31 integrase system is a simple plasmid DNA approach, comprising co-transfection into target cells of a plasmid carrying the attB site and the therapeutic gene, along with a plasmid expressing the integrase. There is no viral vector involved, which eliminates problems associated with viral immunogenicity and toxicity and makes the φC31 integrase system safe to use and inexpensive to manufacture.

Because the φC31 integrase system requires a DNA sequence match with the genome in order to integrate, it uses a much more limited number of integration sites compared to other DNA integration vectors such as transposons and retroviruses, which integrate essentially at random. This is an important safety feature, because random integration can lead to activation of oncogenes.

The φC31 integrase system lacks a size limit, so genes of any size, complete with control regions, can be integrated. For example, φC31 integrase has been used to integrate large plasmids of over 100 kb. This lack of size limit is of particular relevance in treatment of DMD, because the full-length dystrophin cDNA is ˜14 kb long (Koenig, M., et al., 1988, Cell 53:219-228.). Other gene transfer methods such as adeno-associated virus and lentiviral vectors may be unable to carry the full-length dysferlin cDNA.

The safety of using integration sites used by φC31 integrase in human cells is rigorously examined in the other examples provided herein.

That which is claimed is:

1. A method of generating an induced pluripotent stem cell comprising a muscle membrane protein-encoding gene from a cell of a subject, the method comprising:
introducing into the cell isolated from the subject: (i) an expression cassette comprising a polynucleotide encoding a first site-specific unidirectional recombinase and (ii) a first targeting vector comprising a first vector attachment site recognized by the first site-specific unidirectional recombinase, a target site recognized by a second site-specific unidirectional recombinase, and a nucleic acid sequence encoding one or more reprogramming transcription factors, wherein the one or more reprogramming transcription factors induces the cell to form a pluripotent stem cell;
maintaining the cell under conditions sufficient for the first targeting vector to integrate into an endogenous target site in the genome of the cell by a recombination event between the first vector attachment site and the endogenous target site mediated by the first site-specific unidirectional recombinase and to induce the cell to form an induced pluripotent stem cell, wherein the induced pluripotent stem cell comprises the target site;
introducing into the induced pluripotent stem cell: (i) an expression cassette comprising a polynucleotide encoding the second site-specific unidirectional recombinase and (ii) a second targeting vector comprising a second vector attachment site recognized by the second site-specific unidirectional recombinase and a nucleic acid encoding a muscle membrane protein-encoding gene; and
maintaining the induced pluripotent stem cell under conditions sufficient for the second targeting vector to integrate into the target site in the genome of the induced pluripotent stem cell by a recombination event between the second vector attachment site and the target site mediated by the second site-specific unidirectional recombinase to produce an induced pluripotent stem cell comprising the muscle membrane protein-encoding gene.

2. The method of claim 1, wherein the nucleic acid encoding the one or more reprogramming transcription factors is flanked by two compatible targeting sites specific for a bidirectional recombinase, wherein the two compatible targeting sites are arranged in the same orientation.

3. The method of claim 1, wherein the first targeting vector comprises one or more targeting sites specific for a bidirectional recombinase, and wherein the second targeting vector contains one or more targeting sites specific for the same bidirectional recombinase.

4. The method of claim 2, wherein the method comprises excising the nucleic acid encoding the one or more reprogramming transcription factors from the induced pluripotent stem cell by exposing the induced pluripotent stem cell to the bidirectional recombinase, wherein the bidirectional recombinase mediates a recombination event between the two compatible targeting sites.

5. The method of claim 3, wherein the method comprises excising a portion of the first and second targeting vectors from the induced pluripotent stem cell by exposing the induced pluripotent stem cell to the bidirectional recombinase, wherein the bidirectional recombinase mediates a recombination event between a first targeting site on the first targeting vector and a second compatible targeting site on the second targeting vector.

6. The method of claim 1, wherein the cell is a somatic cell.

7. The method of claim 6, wherein the somatic cell is a fibroblast.

8. The method of claim 1, wherein the cell is a multipotent cell.

9. The method of claim 8, wherein the multipotent cell is a mesenchymal stem cell.

10. The method of claim 1, further comprising differentiating the induced stem cell comprising the muscle membrane protein-encoding gene into a muscle cell or a muscle precursor cell.

11. The method of claim 1, further comprising engrafting the induced pluripotent stem cell into the subject.

12. The method of claim 10, further comprising engrafting said muscle cell or said muscle precursor cell into the subject.

13. The method of claim 1, wherein the muscle membrane protein-encoding gene is a dystrophin gene or a dysferlin gene.

14. The method of claim 1, wherein the first and the second site-specific unidirectional recombinases are selected from φC31 integrase, R4 integrase, or Bxb1 integrase.

15. The method of claim 2, wherein the bidirectional recombinase is Cre recombinase.

16. The method of claim 3, wherein the bidirectional recombinase is Cre recombinase.

17. The method of claim 5, wherein the first unidirectional recombinase is φC31 integrase, the second unidirectional recombinase is Bxb1 integrase, and the bidirectional recombinase is Cre recombinase.

18. A method of introducing a dystrophin gene into a subject, the method comprising:
introducing into a fibroblast cell isolated from the subject: (i) a first expression cassette comprising a polynucleotide encoding a first site-specific unidirectional recombinase and (ii) a first targeting vector comprising a nucleic acid encoding a dystrophin gene and a first vector attachment site recognized by the first site-specific unidirectional recombinase;
maintaining the fibroblast cell under conditions sufficient for the targeting vector to integrate into an endogenous target site in the genome of the cell by a recombination event between the first vector attachment site and the endogenous target site mediated by the first site-specific unidirectional recombinase to produce a genetically modified cell; and
engrafting the genetically modified cell in the subject.

19. The method of claim 18, further comprising differentiating the genetically modified cell into a muscle cell or a muscle precursor cell before said engrafting step.

20. The method of claim 18, wherein the first targeting vector comprises a target site recognized by a second site-specific unidirectional recombinase and the target site recognized by a second site-specific unidirectional recombinase is present in the genome of the genetically modified cell, and the method further comprises, before said engrafting step:
introducing into the genetically modified cell: (iii) a second expression cassette encoding the second site-specific unidirectional recombinase and (iv) a second targeting vector comprising a second vector attachment site recognized by the second site-specific unidirectional recombinase and a nucleic acid sequence encoding one or more reprogramming transcription factors, wherein the one or more reprogramming transcription factors induces the genetically modified cell to form a pluripotent stem cell; and
maintaining the genetically modified cell under conditions sufficient for the second targeting vector to integrate into the target site present in the genome of the cell by a recombination event between the second vector attachment site and the target site mediated by the second site-specific unidirectional recombinase and to induce the genetically modified cell to form an induced pluripotent stem cell.

21. The method of claim 20, wherein the nucleic acid encoding the one or more reprogramming transcription factors is flanked by two compatible targeting sites specific for a bidirectional recombinase, wherein the two compatible targeting sites are arranged in the same orientation.

22. The method of claim 20, wherein the first targeting vector comprises a targeting site specific for a bidirectional recombinase, and wherein the second targeting vector comprises a targeting site specific for the same bidirectional recombinase.

23. The method of claim 21, wherein the method comprises excising the nucleic acid encoding the one or more reprogramming transcription factors from the induced pluripotent stem cell by exposing the induced pluripotent stem cell to a site specific bidirectional recombinase, wherein the bidirectional recombinase mediates a recombination event between the two compatible targeting sites.

24. The method of claim 22, wherein the method further comprises excising a portion of the first and second targeting vectors from the induced pluripotent stem cell by exposing the induced pluripotent stem cell to the bidirectional recombinase, wherein the bidirectional recombinase mediates a recombination event between the first targeting site on the first targeting vector and the second targeting site on the second targeting vector.

25. The method of claim 19, wherein the subject is a human diagnosed with Duchenne muscular dystrophy.

26. The method of claim 18, wherein the first site-specific unidirectional recombinase is φC31 integrase.

27. The method of claim 20, wherein the second site-specific unidirectional recombinase is Bxb1 integrase.
